SEQUENCE LISTING (16 sequences)

SEQ ID NO: 1 - protein, 10 aa, synthetic construct
ADIYTPEVGR

SEQ ID NO: 2 - protein, 12 aa, synthetic construct
GQVQVVDNFGNR

SEQ ID NO: 3 - protein, 7 aa, synthetic construct
QFEEQQR

SEQ ID NO: 4 - protein, 11 aa, synthetic construct
ELYETASELPR

SEQ ID NO: 5 - protein, 9 aa, synthetic construct
LNALEPTNR

SEQ ID NO: 6 - protein, 10 aa, synthetic construct
ADIYTEQVGR

SEQ ID NO: 7 - protein, 9 aa, synthetic construct
LLSGIENFR

SEQ ID NO: 8 - protein, 9 aa, synthetic construct
GNIVNEFER

SEQ ID NO: 9 - protein, 9 aa, synthetic construct
HESEEGEVK

SEQ ID NO: 10 - protein, 8 aa, synthetic construct
FFDQQEQR

SEQ ID NO: 11 - protein, 9 aa, synthetic construct
LDALEPTNR

SEQ ID NO: 12 - protein, 10 aa, synthetic construct
ADIYTEEAGR

SEQ ID NO: 13 - protein, 8 aa, synthetic construct
RPSHQQPR

SEQ ID NO: 14 - protein, 16 aa, synthetic construct
IFLAGDKDNVIDQIEK

SEQ ID NO: 15 - protein, 9 aa, synthetic construct
QQWELQGDR

SEQ ID NO: 16 - protein, 9 aa, synthetic construct
DLEVESGGR

(19) United States
(12) Patent Application Publication
(10) Pub. No.: US 2026/0216320 A1
(43) Pub. Date: Jul. 30, 2026

(54) MIXED NUT ALLERGEN FORMULATIONS AND METHODS OF MAKING AND USING THE SAME

(71) Applicant: SOCIÉTÉ DES PRODUITS NESTLÉ S.A., Vevey (CH)

(72) Inventors: Abhi KATHURIA, San Diego, CA (US); Jonathan James GILROY, Everett, WA (US)

(21) Appl. No.: 19/145,761

(22) PCT Filed: Jan. 17, 2024
(86) PCT No.: PCT/EP2024/051066

§ 371 Date: Jul. 3, 2025

Related U.S. Application Data
(60) Provisional application No. 63/439,395, filed on Jan. 17, 2023.

(51) Int. Cl. A61K 39/35; A61K 9/16; A61K 39/00

(52) U.S. Cl. CPC A61K 39/35; A61K 9/1611; A61K 9/1617; A61K 9/1623; A61K 9/1652; A61K 9/1694; A61K 2039/577

(57) ABSTRACT

The present disclosure provides, in part, compositions comprising mixed allergen powders of one or more nut flours and one or more excipients, methods of producing the compositions, and methods of using the same for the prevention and treatment of nut allergies.

### CROSS-REFERENCE TO RELATED APPLICATIONS

This application claims the benefit of and priority to U.S. Provisional Application No. 63/439,395, filed Jan. 17, 2023, the entire contents of which are herein incorporated by reference.

### FIELD

The present disclosure provides, in part, formulations of mixed nut allergens and methods of making and using the same, e.g., for desensitization of nut allergies.

### SEQUENCE LISTING

The instance application contains a sequence listing, which has been submitted in XML format. The contents of the XML file, named “106672-01-5013-PC_Sequence_Listing.xml,” which was created on Jan. 6, 2024, and is 14,959 bytes in size, are incorporated herein by reference in their entirety.

### BACKGROUND

Allergies affect humans and companion animals, and some allergic reactions (for example, those to insects, foods, latex, and drugs) can be so severe as to be life threatening.

Allergic reactions result when a subject's immune system responds to an allergen. Typically, there is no allergic reaction the first time a subject is exposed to a particular allergen. However, it is the initial response to an allergen that primes the system for subsequent allergic reactions. In particular, the allergen is taken up by antigen presenting cells (APCs; e.g., macrophages and dendritic cells) that degrade the allergen and then display allergen fragments to T-cells. T-cells, in particular CD4+ “helper” T-cells, respond by secreting a collection of cytokines that have effects on other immune system cells. The profile of cytokines secreted by responding CD4+ T-cells determines whether subsequent exposures to the allergen will induce allergic reactions. Two classes of CD4+ T-cells (Th1 and Th2, T-lymphocyte helper type) influence the type of immune response that is mounted against an allergen.

The Th1-type immune response involves the stimulation of cellular immunity to allergens and infectious agents and is characterized by the secretion of IL-2, IL-6, IL-12, IFN-gamma, and TNF-beta by CD4+T helper cells and the production of IgG antibodies. Exposure of CD4+ T-cells to allergens can also activate the cells to develop into Th2 cells, which secrete IL-4, IL-5, IL-10, and IL-13. IL-4 production stimulates maturation of B cells that produce IgE antibodies specific for the allergen. These allergen-specific IgE antibodies attach mast cell and basophil receptors, where they initiate a rapid immune response to the next exposure to allergen. When the subject encounters the allergen a second time, the allergen is quickly bound by these surface-associated IgE molecules, resulting in the release of histamines and other substances that trigger allergic reactions. Subjects with high levels of IgE antibodies are known to be particularly prone to allergies.

There remains a need for compositions for the desensitization of allergies, particularly food allergies, which are manufacturable into formulations suitable for oral immunotherapy (OIT).

### SUMMARY

Without being bound by any theory, the present inventors believe that an excipient having a high surface area and/or high diameter can accommodate more active agent and provide improved flow properties of a mixed allergen powder comprising at least one tree nut flour.

Accordingly, the present disclosure provides a mixed allergen powder contains tree nut flours and additionally contains an excipient. The excipient has a particle size up to about 150 microns, for example, no greater than about 140 microns, no greater than about 130 microns, no greater than about 120 microns, no greater than about 110 microns, or no greater than about 100 microns. Each of the tree nut flours may have a particle size up to about 100 microns, for example, no greater than about 75 microns, no greater than about 50 microns, or no greater than about 40 microns.

The excipient may include at least one of 6-O-a-D-glucopyranosyl-D-sorbitol (1,6-GPS), 1-O-a-D-glucopyranosyl-D-mannitol (1,1-GPM), D-mannitol. The excipient may include a texturizer, such as one or more of microcrystalline cellulose, guar gum, or mannitol. In some embodiments, the texturizer comprises at least one of AVICEL® CE15 or AVICEL® HFE 102.

Also, a method of making a mixed allergen powder includes mixing a plurality of tree nut flours with an excipient; and another method of making a mixed allergen powder includes providing an excipient that has been spray-dried; and mixing a plurality of tree nut flours with the excipient. A method of preventing or treating at least one tree nut allergy in a subject, preferably by oral immunotherapy, includes administering the mixed allergen powder to the subject. In each of these methods, the excipient has a particle size up to about 150 microns, for example, no greater than about 140 microns, no greater than about 130 microns, no greater than about 120 microns, no greater than about 110 microns, or no greater than about 100 microns. Each of the tree nut flours may have a particle size up to about 100 microns, for example, no greater than about 75 microns, no greater than about 50 microns, or no greater than about 40 microns.

In embodiments, disclosed herein are compositions comprising nut allergen powders which have a high surface area and/or high diameter that accommodate more active agent (e.g., allergenic peptide) and provide improved flow properties of a mixed allergen powder (e.g., cashew, hazelnut, walnut, and/or peanut) that is conducive to formulation into oral immunotherapy (OIT) doses, e.g., capsules and/or sachets.

In embodiments, the mixed allergen powder comprises one or more nut flours, where the one or more nut flours comprises cashew flour, hazelnut flour, walnut flour, peanut flour, and combinations thereof.

In embodiments, the cashew flour comprises about or at least about 25% to about 40% protein content, optionally about 32% protein content, the hazelnut flour comprises about or at least about 30% to about 45% protein content, optionally about 38% protein content, the walnut flour comprises about or at least about 35% to about 55% protein content, optionally about 47% protein content, and the peanut flour comprises about or at least about 40% to about 60% protein content, optionally about 50% protein content.

In embodiments, the one or more nut flours comprise defatted nut flour, optionally about or at least about 10%, 12%, or 15% defatted nut flour.

In embodiments, the composition comprises one or more of about or at least about 1% (w/w) to about 25% (w/w) cashew flour, about or at least about 1% (w/w) to about 25% (w/w) hazelnut flour, about or at least about 1% (w/w) to about 25% (w/w) walnut flour, about or at least about 1% (w/w) to about 25% (w/w) peanut flour.

In embodiments, the composition comprises cashew flour present in about 20%±1% (w/w) per 1200 mg of composition, hazelnut flour present in about 17%±1% (w/w) per 1200 mg of composition; walnut flour present in about 14%±1% (w/w) per 1200 mg composition, and peanut flour present in about 12%±1% (w/w) per 1200 mg of composition.

In embodiments, the composition comprises cashew flour present in about 10%±1% (w/w) per 80 mg of composition, hazelnut flour present in about 10%±1% (w/w) per 80 mg of composition; walnut flour present in about 4%±1% (w/w) per 80 mg composition, and peanut flour present in about 1%±0.5% (w/w) per 80 mg of composition.

In embodiments, the composition comprises cashew flour present in about 2%±1% (w/w) per 0.8 mg of composition, hazelnut flour present in about 1.5%±1% (w/w) per 0.8 mg of composition; walnut flour present in about 1%±0.5% (w/w) per 0.8 mg composition, and peanut flour present in about 1%±0.5% (w/w) per 0.8 mg of composition.

In embodiments, the one or more nut allergen peptides comprise 2S albumin (Ana) protein, 11S globulin (Cor) protein, 7S globulin (Jug) protein, and 2S albumin (Arah) protein.

In embodiments, the one or more nut allergen peptides comprises an amino acid sequence of SEQ ID NO: 1 from an Ana o 2-292 protein, an amino acid sequence of SEQ ID NO: 2 from an Ana o 2-350 protein, an amino acid sequence of SEQ ID NO: 3 from an Ana o 3-042 protein, and/or an amino acid sequence of SEQ ID NO: 4 from an Ana o 3-112 protein, each of which being derived from a cashew.

In embodiments, the one or more nut allergen peptides comprises an amino acid sequence of SEQ ID NO: 5 from a Cor a 9-043 protein, an amino acid sequence of SEQ ID NO: 6 from a Cor a 9-341 protein, an amino acid sequence of SEQ ID NO: 7 from a Cor a 11-092 protein, and/or an amino acid sequence of SEQ ID NO: 8 from a Cor a 11-395 protein, each of which being derived from a hazelnut.

In embodiments, the one or more nut allergen peptides comprises an amino acid sequence of SEQ ID NO: 9 from a Jug r 2-193 protein, an amino acid sequence of SEQ ID NO: 10 from a Jug r 2-343 protein, an amino acid sequence of SEQ ID NO: 11 from a Jug r 4-041 protein, and/or an amino acid sequence of SEQ ID NO: 12 from a Jug r 4-336 protein, each of which being derived from a walnut.

In embodiments, the one or more nut allergen peptides comprises an amino acid sequence of SEQ ID NO: 13 from an Ana h 1-128 protein, an amino acid sequence of SEQ ID NO: 14 from an Ana h 1-541 protein, an amino acid sequence of SEQ ID NO: 15 from an Ana h 2-023 protein, an amino acid sequence of SEQ ID NO: 16 from an Ana h 2-160 protein, each of which being derived from a peanut.

In embodiments, the one or more nut allergen peptides comprise the amino acid sequences of SEQ ID NOs: 1-16. In embodiments, the one or more nut allergen peptides comprise one or more modifications, e.g., carbamidomethylation.

In embodiments, the one or more excipients are present in a total amount of about 1% (w/w) to about 95% (w/w). In embodiments, the mixed allergen powder particle size less than about 90 μm, less than about 80 μm, less than about 70 μm, less than about 60 μm, less than about 50 μm, less than about 40 μm.

In embodiments, the one or more excipients comprises 6-O-α-D-glucopyranosyl-D-sorbitol (1,6-GPS), 1-O-α-D-glucopyranosyl-D-mannitol (1,1-GPM) (isomalt), mannitol, sorbitol, sucrose, maltodextrin, lactose, microcrystalline cellulose, guar gum, starch (corn starch and/or pregelatinized starch), micronized talc, magnesium stearate, silica (fumed silica, silica dioxide), and combinations thereof.

In embodiments, the composition comprises STARCAP 1500 (corn starch and pregelatinized starch), AVICEL PH-101 (partially depolymerized microcrystalline alpha-cellulose), AVICEL HFE-102 (e.g., microcrystalline cellulose and mannitol), AVICEL CE-15 (e.g., microcrystalline cellulose and guar gum), CAB-O-SIL M-5P (fumed silica), PARTEK SI 150 (sorbitol), PARTEK M200 (spray-dried mannitol), GALENIQ 720, (agglomerated spherical isomalt of spray-dried sorbitol/mannitol), PEARLITOL SD200 (mannitol), FASTFLO 316 (spray-dried mixture of crystalline and amorphous lactose), and/or DI-PAC (granulated sugar, maltodextrin). The excipient may include at least one of 6-O-α-D-glucopyranosyl-D-sorbitol (1,6-GPS), 1-O-α-D-glucopyranosyl-D-mannitol (1,1-GPM), D-mannitol.

In embodiments, the composition has an average angle of repose of about or less than about 45 degrees, an average height of cone according to an angle of repose analysis of between about 5 cm to about 7 cm, and/or an average base of cone according to an angle of repose analysis of about 12 cm to about 14 cm, a Carr's Index of about 30 to about 60, or about 35 to about 55, or about 40 to about 50, and/or a bulk density of about 0.25 g/mL to about 0.35 g/mL; and/or a tapped density of about 0.5 g/mL to about 0.7 g/mL.

In embodiments, the composition comprises a mixed allergen powder comprising cashew flour, hazelnut flour, walnut flour, and peanut flour, wherein the mixed allergen powder has a particle size of less than about 100 μm, and one or more excipients comprising pregelatinized starch, microcrystalline cellulose, guar gum, agglomerated spherical isomalt, mannitol, sorbitol, sucrose, lactose, maltodextrin, fumed silica, micronized talc, and magnesium stearate, wherein the one or more excipients has a particle size up to about 150 μm.

In embodiments, the composition consists of a mixed allergen powder consisting of cashew flour, hazelnut flour, walnut flour, and peanut flour, wherein the mixed allergen powder has a particle size of less than about 100 μm, and one or more excipients selected from pregelatinized starch, microcrystalline cellulose, guar gum, agglomerated spherical isomalt, mannitol, sorbitol, sucrose, lactose, maltodextrin, fumed silica, micronized talc, and magnesium stearate, wherein the one or more excipients has a particle size up to about 150 μm.

In aspects, disclosed herein is a method of making composition, e.g., as described herein, the method comprising mixing one or more nut flours with one or more excipients, where the one or more nut flours each have been processed to have a particle size of less than about 100 μm, and where the one or more excipients each have been processed to have a particle size of less than about 150 μm.

Also, a method of making a mixed allergen powder includes mixing a plurality of tree nut flours with an excipient; and another method of making a mixed allergen powder includes providing an excipient that has been spray-dried; and mixing a plurality of tree nut flours with the excipient. A method of preventing or treating at least one tree nut allergy in a subject, preferably by oral immunotherapy, includes administering the mixed allergen powder to the subject. In each of these methods, the excipient has a particle size up to about 150 microns, for example, no greater than about 140 microns, no greater than about 130 microns, no greater than about 120 microns, no greater than about 110 microns, or no greater than about 100 microns. Each of the tree nut flours may have a particle size up to about 100 microns, for example, no greater than about 75 microns, no greater than about 50 microns, or no greater than about 40 microns.

In embodiments, the method comprises defatting the one or more nut flours to form one or more nut flour cakes, optionally wherein the defatting reduces the fat content to less than about 12% (w/w) prior to the mixing with the one or more excipients. In embodiments, the methods comprising milling the one or more nut flour cakes to a predetermined particle size prior to the mixing with the one or more excipients. In embodiments, the milling forms a uniform, free-flowing blend of the one or more nut flours, optionally as determined by one or more of an angle of repose analysis, Carr's index, or a FLODEX analysis (e.g., sometimes referred to as a FLOWDEX analysis). In embodiments, the method comprises determining the protein content of the one or more nut flours and/or controlling the protein content of the one or more nut flours prior to the mixing. In embodiments, the method formulating the mixed allergen powder into a form suitable for oral administration, optionally capsules or sachets.

In aspects, described herein is a method of preventing or treating one or more nut allergy in a subject in need thereof, the method comprising administering a composition or pharmaceutical composition, e.g., as described herein, where the subject has one or more of a cashew allergy, hazelnut allergy, walnut allergy, and peanut allergy.

### BRIEF DESCRIPTION OF THE DRAWINGS

FIGS. 1A-1B provide results of a sieve analysis for PALFORZIA. The percent retained (FIG. 1A) and the particle size (FIG. 1B) of PALFORZIA (oral immunotherapy peanut allergen) are shown. For both of these figures, the units for the x-axis are microns.

FIGS. 2A-2B provide results of a sieve analysis formulation 21003-17 (milled cashew). The percent retained (FIG. 2A) and the particle size (FIG. 2B) of formulation 21003-17 (milled cashew) are shown. For both of these figures, the units for the x-axis are microns.

FIGS. 3A-3B provide results of a sieve analysis formulation 21003-20 (milled hazelnut). The percent retained (FIG. 3A) and the particle size (FIG. 3B) of formulation 21003-20 (milled hazelnut) are shown. For both of these figures, the units for the x-axis are microns.

FIGS. 4A-4B provide results of a sieve analysis formulation 21003-23 (milled walnut). The percent retained (FIG. 4A) and the particle size (FIG. 4B) of formulation 21003-23 (milled walnut) are shown. For both of these figures, the units for the x-axis are microns.

FIG. 5 shows a schematic illustrating the sampling plan for the blend uniformity assays in accordance with some embodiments.

### DETAILED DESCRIPTION

As used in this disclosure and the appended claims, the singular forms “a,” “an” and “the” include plural referents unless the context clearly dictates otherwise. Thus, for example, reference to “a flour” or “the flour” includes a single flour and also two or more flours.

The words “comprise,” “comprises,” and “comprising” are to be interpreted inclusively rather than exclusively. Likewise, in embodiments, the terms “include,” “including,” and “or,” are construed to be inclusive, unless such a construction is clearly prohibited from the context. Thus, in embodiments, a disclosure of an embodiment using the term “comprising” includes a disclosure of embodiments “consisting essentially of” and “consisting of” the components identified. Similarly, in embodiments, the methods disclosed herein may lack any step that is not specifically disclosed herein. Thus, in embodiments, a disclosure of an embodiment using the term “comprising” includes a disclosure of embodiments “consisting essentially of” and “consisting of” the steps identified. Any embodiment disclosed herein can be combined with any other embodiment disclosed herein.

The terms “at least one of,” and “and/or” used respectively in the context of “at least one of X or Y” and “X and/or Y” should be interpreted as “X without Y,” or “Y without X,” or “both X and Y.” In embodiments, where used herein, the terms “example” and “such as,” particularly when followed by a listing of terms, are merely exemplary and illustrative and should not be deemed to be exclusive or comprehensive.

All percentages expressed herein are by weight of the total weight of the composition unless expressed otherwise. As used herein, in embodiments, the term “about” is understood to refer to numbers in a range of numerals, for example the range of −10% to +10% of the referenced number.

All numerical ranges herein include all integers, whole numbers, or fractions, within the range. For example, in embodiments, a disclosure of from 1 to 10 should be construed as supporting a range of from 1 to 8, from 3 to 7, from 1 to 9, from 3.6 to 4.6, from 3.5 to 9.9, and so forth.

Any embodiment disclosed herein can be combined with any other embodiment disclosed herein unless explicitly stated otherwise.

The term “allergen,” as used herein, refers to a subset of antigens which elicit the production of IgE in addition to other isotypes of antibodies. Preferred allergens for the present disclosure are protein allergens.

As used herein, “particle size” means average diameter in microns (μm).

“Prevention” includes reduction of risk, incidence and/or severity of a condition or disorder, e.g., oral desensitization with respect to nut peptide allergens. The terms “treatment” and “treat” include both prophylactic or preventive treatment (e.g., that prevents and/or slows the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery, or that an allergy disappears. The terms “treatment” and “treat” refer to the maintenance and/or promotion of health in an individual not suffering from a disease (e.g., allergy) but who may be susceptible to the development of an unhealthy condition, e.g., adult-onset allergies or food intolerances. The terms “treatment” and “treat” also include the potentiation or otherwise enhancement of one or more primary prophylactic or therapeutic measures, e.g., reducing the reliance on and/or probability of needing epinephrine for allergy. As non-limiting examples, a treatment can be performed by a patient, a caregiver, a doctor, a nurse, or another healthcare professional.

As used herein, a prophylactically or therapeutically “effective amount” is an amount that prevents a deficiency, treats a disease or medical condition in an individual, or, more generally, reduces symptoms, manages progression of the disease, or provides a nutritional, physiological, or medical benefit to the individual.

Preferred embodiments provided by the present disclosure are described hereafter.

An aspect of the present disclosure is a mixed allergen powder comprising a plurality of tree nut flours and further comprising an excipient. The excipient has a particle size up to about 150 microns, for example, no greater than about 140 microns, no greater than about 130 microns, no greater than about 120 microns, no greater than about 110 microns, or no greater than about 100 microns. Each of the tree nut flours may have a particle size up to about 100 microns, for example, no greater than about 75 microns, no greater than about 50 microns, or no greater than about 40 microns.

In some embodiments, the plurality of tree nut flours comprises at least one tree nut flour selected from the group consisting of cashew flour, hazelnut flour, walnut flour, peanut flour, and mixtures thereof, preferably at least two thereof, more preferably at least three thereof, and most preferably all four thereof.

In some embodiments, the excipient comprises at least one of 6-O-α-D-glucopyranosyl-D-sorbitol (1,6-GP S) and/or 1-O-α-D-glucopyranosyl-D-mannitol (1,1-GPM), preferably both 1,6-GPS and 1,1-GPM, more preferably both 1,6-GPS and 1,1-GPM in an approximately 1:1 ratio and/or in isomalt included in the mixed allergen powder. In some embodiments, the 1,6-GPS and/or the 1,1-GPM is agglomerated, preferably in isomalt that is agglomerated.

In some embodiments, the excipient comprises D-mannitol.

In some embodiments, the excipient comprises a texturizer. Preferably, the texturizer comprises microcrystalline cellulose and optionally further comprises guar gum, for example the microcrystalline cellulose and the guar gum in an approximately 17:3 ratio. The texturizer may further comprise mannitol, for example the microcrystalline cellulose and the mannitol in an approximately 9:1 ratio. In some embodiments, the texturizer comprises at least one of AVICEL® CE15 or AVICEL® HFE 102.

In some embodiments, the mixed allergen powder further comprises starch, preferably corn starch. Preferably, at least a portion of the starch is pregelatinized. In some embodiments, the starch comprises approximately 85-95% corn starch and approximately 5-15% pregelatinized starch.

In some embodiments, the mixed allergen powder further comprises fumed silica, preferably fumed silica having a bulk density of about 60 g/L or less.

In some embodiments, the mixed allergen powder further comprises magnesium stearate.

In some embodiments, at least 100 g of the powder passes through a 32 mm discharge hole in about 20 seconds. The powder may have a Carr's index less than 45 and/or an average angle of repose less than 45.

Another aspect of the present disclosure is a method of making a mixed allergen powder, the method comprising: mixing a plurality of tree nut flours with an excipient. Yet another aspect of the present disclosure is a method of making a mixed allergen powder, the method comprising: providing an excipient that has been spray-dried; and mixing a plurality of tree nut flours with the excipient. In each of these methods, the excipient has a particle size up to about 150 microns, for example, no greater than about 140 microns, no greater than about 130 microns, no greater than about 120 microns, no greater than about 110 microns, or no greater than about 100 microns. Each of the tree nut flours may have a particle size up to about 100 microns, for example, no greater than about 75 microns, no greater than about 50 microns, or no greater than about 40 microns.

In some embodiments, the method comprises defatting at least one of the plurality of tree nut flours to form a cake, preferably having a fat content of less than 12 wt. %, before mixing the plurality of tree nut flours with the excipient. The method may further comprise milling the cake to a predetermined particle size before mixing the plurality of tree nut flours with the excipient. The milling preferably forms a uniform, free-flowing blend of the plurality of tree nut flours. In some embodiments, the method further comprises filling the mixed allergen powder into capsules.

Another aspect of the present disclosure is a method of preventing or treating at least one tree nut allergy in a subject, preferably by oral immunotherapy, the method comprising administering any mixed allergen powder disclosed herein and/or a mixed allergen powder made by any method disclosed herein to the subject.

The compositions described herein can be formulated for administration to a subject by any conventional means including, but not limited to, oral administration routes. As used herein, the term “subject” is used to mean an animal, preferably a mammal, including a human or nonhuman. The terms subject and subject may be used interchangeably. The formulations are for prevention and treatment of symptoms associated with exposure to limited amounts of peanut allergen in children and adults. In some embodiments, a subject is from about 4 to about 26 years of age.

### Compositions and Formulations of Mixed Nut Allergens

In embodiments, described herein are compositions comprising mixed allergen powder comprising one or more nut allergen peptides, wherein the mixed allergen powder comprises a particle size of less than about 100 μm. In embodiments, the compositions comprise one or more excipients comprising a particle size up to about 150 μm. The particle sizes, in embodiments, provide bulk properties that are conducive to the manipulation necessary to formulate the powders into therapeutic compositions (e.g., capsules or sachets).

In embodiments, the mixed allergen powder comprises tree nut flours (e.g., cashew, hazelnut, and/or walnut), and/or legume nut powder (e.g., peanut). In embodiments, the composition comprises combinations of cashew flour, hazelnut flour, walnut flour, and peanut flour.

In some embodiments, the plurality of tree nut flours comprises at least one tree nut flour selected from the group consisting of cashew flour, hazelnut flour, walnut flour, peanut flour, and mixtures thereof, preferably at least two thereof, more preferably at least three thereof, and most preferably all four thereof.

In embodiments, the cashew flour comprises about or at least about 25% to about 40% protein content, optionally about 32% protein content. In embodiments, the hazelnut flour comprises about or at least about 30% to about 45% protein content, optionally about 38% protein content. In embodiments, the walnut flour comprises about or at least about 35% to about 55% protein content, optionally about 47% protein content. In embodiments, the peanut flour comprises about or at least about 40% to about 60% protein content, optionally about 50% protein content.

In embodiments, the protein content of nut flours is measurable by, for example, HPLC methods (chromatography to determine retention volumes relative to a protein standard), enzyme-linked immunosorbent assays (ELISA; using antibodies which bind specific proteins/allergens), gel electrophoresis (SDS-PAGE), Western blotting and immunoprecipitation, and/or liquid chromatography mass spectroscopy (LC-MS/MS). Persons skilled in the art will recognize the numerous methods useful to determine protein contents of samples. In embodiments, the protein content of nut flours comprise a purity factor, for example, of about or at least about 0.3 to about or at least about 0.5.

In embodiments, the one or more nut flours comprise defatted nut flour. In embodiments, the nut flour is about or at least about 10%, 12%, or 15% defatted. In embodiments, the one or more nut flours originate from shelled, raw nuts, which have been roasted and partially defatted, having about 12% (w/w) fat content.

In embodiments, the composition comprises one or more of about or at least about 1% (w/w) to about 25% (w/w) cashew flour, about or at least about 1% (w/w) to about 25% (w/w) hazelnut flour, about or at least about 1% (w/w) to about 25% (w/w) walnut flour, and about or at least about 1% (w/w) to about 25% (w/w) peanut flour.

In embodiments, the composition comprises or consists of cashew flour present in about 20% (e.g., ±1%) (w/w) per 1200 mg of composition, hazelnut flour present in about 17% (e.g., ±1%) (w/w) per 1200 mg of composition; walnut flour present in about 14% (e.g., ±1%) (w/w) per 1200 mg composition, and peanut flour present in about 12% (e.g., ±1%) (w/w) per 1200 mg of composition.

In embodiments, the composition comprises or consists of cashew flour present in about 10% (e.g., ±1%) (w/w) per 80 mg of composition, hazelnut flour present in about 10% (e.g., ±1%) (w/w) per 80 mg of composition; walnut flour present in about 4% (e.g., ±1%) (w/w) per 80 mg composition, and peanut flour present in about 1% (e.g., ±0.5%) (w/w) per 80 mg of composition.

In embodiments, the composition comprises or consists of cashew flour present in about 2% (e.g., ±1%) (w/w) per 0.8 mg of composition, hazelnut flour present in about 1.5% (e.g., ±1%) (w/w) per 80 mg of composition; walnut flour present in about 1% (e.g., ±0.5%) (w/w) per 80 mg composition, and peanut flour present in about 1% (e.g., ±0.5%) (w/w) per 80 mg of composition.

In embodiments, the one or more nut allergen peptides present in the one or more nut flours comprise 2S albumin (Ana) protein (e.g., from cashew), 11S globulin (Cor) protein (e.g., from hazelnut), 7S globulin (Jug) protein (e.g., from walnut), and 2S albumin (Arah) protein (e.g., from peanut).

In embodiments, the mixed allergen powder comprises one or more allergenic peptides, as indicated in Table 1.

In embodiments, the one or more nut allergen peptides comprises an amino acid sequence of SEQ ID NO: 1 from an Ana o 2-292 protein, an amino acid sequence of SEQ ID NO: 2 from an Ana o 2-350 protein, an amino acid sequence of SEQ ID NO: 3 from an Ana o 3-042 protein, and/or an amino acid sequence of SEQ ID NO: 4 from an Ana o 3-112 protein, each of which being derived from a cashew (e.g., Anacardium occidentale).

In embodiments, the one or more nut allergen peptides comprises an amino acid sequence of SEQ ID NO: 5 from a Cor a 9-043 protein, an amino acid sequence of SEQ ID NO: 6 from a Cor a 9-341 protein, an amino acid sequence of SEQ ID NO: 7 from a Cor a 11-092 protein, and/or an amino acid sequence of SEQ ID NO: 8 from a Cor a 11-395 protein, each of which being derived from a hazelnut (e.g., Corylus avellana).

In embodiments, the one or more nut allergen peptides comprises an amino acid sequence of SEQ ID NO: 9 from a Jug r 2-193 protein, an amino acid sequence of SEQ ID NO: 10 from a Jug r 2-343 protein, an amino acid sequence of SEQ ID NO: 11 from a Jug r 4-041 protein, and/or an amino acid sequence of SEQ ID NO: 12 from a Jug r 4-336 protein, each of which being derived from a walnut (e.g., Juglans regia).

In embodiments, the one or more nut allergen peptides comprises an amino acid sequence of SEQ ID NO: 13 from an Ana h 1-128 protein, an amino acid sequence of SEQ ID NO: 14 from an Ana h 1-541 protein, an amino acid sequence of SEQ ID NO: 15 from an Ana h 2-023 protein, and/or an amino acid sequence of SEQ ID NO: 16 from an Ana h 2-160 protein, each of which being derived from a peanut (e.g., Arachis hypogaea).

In embodiments, the one or more nut allergen peptides comprise, or consist of, the amino acid sequences of one or more of SEQ ID NOs: 1-16; in some embodiments, each of SEQ ID NOs: 1-16.

In embodiments, the one or more nut allergen peptides comprise one or more modifications (e.g., post-translational modification), including for example carbamidomethylation, conjugation and/or crosslinking with e.g., glutaraldehyde, or carbamylation with cyanates. In embodiments, the one or more modifications includes any modification useful for allergenic peptides for the purposes of oral desensitization.

In embodiments, the compositions further comprising one or more excipients. In embodiments, the one or more excipients have a particle size up to about 150 microns, for example, no greater than about 140 microns, no greater than about 130 microns, no greater than about 120 microns, no greater than about 110 microns, or no greater than about 100 microns. Each of the tree nut flours may have a particle size up to about 100 microns, for example, no greater than about 75 microns, no greater than about 50 microns, or no greater than about 40 microns.

In embodiments, the one or more excipients comprises 6-O-α-D-glucopyranosyl-D-sorbitol (1,6-GPS), 1-O-α-D-glucopyranosyl-D-mannitol (1,1-GPM) (isomalt; spray-dried GALENIQ), mannitol, sorbitol (e.g., PARTECK SI 150 (sorbitol) or PARTECK M200 (mannitol)), sucrose, maltodextrin (e.g., DI-PAC granulated sugar, maltodextrin), microcrystalline cellulose (e.g., STARCAP), guar gum, starch (corn starch and/or pregelatinized starch), magnesium stearate, silica (fumed silica, silica dioxide), and combinations thereof.

In some embodiments, the excipient comprises at least one of 6-O-α-D-glucopyranosyl-D-sorbitol (1,6-GP S) and/or 1-O-α-D-glucopyranosyl-D-mannitol (1,1-GPM), preferably both 1,6-GPS and 1,1-GPM, more preferably both 1,6-GPS and 1,1-GPM in an approximately 1:1 ratio and/or in isomalt included in the mixed allergen powder. In some embodiments, the 1,6-GPS and/or the 1,1-GPM is agglomerated, preferably in isomalt that is agglomerated.

In embodiments, the excipient comprises at least one of 6-O-α-D-glucopyranosyl-D-sorbitol (1,6-GP S) and/or 1-O-α-D-glucopyranosyl-D-mannitol (1,1-GPM), and/or both 1,6-GPS and 1,1-GPM, and/or both 1,6-GPS and 1,1-GPM in an approximately a 1:1 ratio to 1:5 ratio, or to a 5:1 ratio, and/or in isomalt included in the mixed allergen powder. In embodiments, the 1,6-GPS and/or the 1,1-GPM is agglomerated, preferably in isomalt that is agglomerated.

In embodiments, the excipient comprises D-mannitol.

In embodiments, the excipient comprises a texturizer. Preferably, the texturizer comprises microcrystalline cellulose and optionally further comprises guar gum, for example the microcrystalline cellulose and the guar gum in an approximately 17:3 ratio. The texturizer may further comprise mannitol, for example the microcrystalline cellulose and the mannitol in an approximately 9:1 ratio. In some embodiments, the texturizer comprises at least one of AVICEL CE15 or AVICEL HFE 102.

In embodiments, the mixed allergen powder further comprises starch, preferably corn starch. Preferably, at least a portion of the starch is pregelatinized. In some embodiments, the starch comprises approximately 85-95% corn starch and approximately 5-15% pregelatinized starch.

In embodiments, the mixed allergen powder further comprises magnesium stearate.

In embodiments, the composition comprises STARCAP 1500, which is a co-processed mixture of globally accepted excipients, corn starch, and pregelatinized starch, with a bulk density of approximately 0.42, a tapped density of 0.58, and a median particle diameter (D50) of ~90 μm. In embodiments, the shape and size of STARCAP 1500 results in improved flow over similarly sized microcrystalline cellulose (MCC) alone. Moreover, in embodiments, STARCAP 1500 is inert, improves stability, provides weight uniformity due to its excellent flow in high speed capsule filling operation, and starts to dissolve is water by acting as a binder, thus aiding in mouthfeel upon ingestion.

In embodiments, the composition comprises AVICEL PH-101, which is a purified, partially depolymerized microcrystalline alpha-cellulose made by acid hydrolysis of specialty wood pulp, with a D50 of ~50 μm. In embodiments, the composition comprises AVICEL HFE-102 (e.g., microcrystalline cellulose and mannitol) and/or AVICEL CE-15 (e.g., microcrystalline cellulose and guar gum). In embodiments, AVICEL HFE-102 is a spray-dried blend comprised of 90% microcrystalline cellulose (MCC) and 10% mannitol, with a bulk density of ~0.41, a tapped density of ~0.51, and a D50 of ~100-130 μm. In embodiments, the mannitol/MCC combination improves palatability, decreases the disintegration time due to the good water solubility and wetting properties, and the mannitol confers a negative heat of solution. In embodiments, AVICEL CE-15 is a spray-dried blend comprising 85% MCC and 15% guar gum, with a bulk density of ~0.5, a tapped density of ~0.62, and a D50 of ~100 μm. In embodiments, it improves sensory attributes by reducing grittiness, tooth packing, and friability to create a creamier mouthfeel, while being highly hygroscopic and weakly acidic.

In embodiments, the composition comprises CAB-O-SIL M-5P, which is a fumed silica that is compatible with many pharmaceutical ingredients, and has a particle surface area of 200 m²/g and a bulk density of <60 g/L. In embodiments, it adheres readily to hydrophilic ingredients, functioning as an excellent glidant, and is easy to disperse.

In embodiments, the composition comprises PARTEK SI 150, which is a sorbitol that is compatible with many pharmaceutical ingredients, and has a D50 of ~1.49 g/cm³, a bulk density of ~450 kg/cm³, and a solubility of ~2350 g/L. In embodiments, it is a directly compressible sorbitol with an excellent tableting behavior and a pleasant mouthfeel and taste; moreover, it enables fast disintegration and dissolution, even at high tablet hardness, and it is well-suitable for low dose API formulations as it supports a good content uniformity.

In embodiments, the composition comprises PARTEK M200, which is a spray-dried mannitol that is compatible with many pharmaceutical ingredients, and has a D50 of ~1.52 g/cm³, a bulk density of ~400-500 kg/cm³, and a solubility of ~213 g/L. In embodiments, it is specifically for solid oral dose applications, and is a directly compressible mannitol with excellent compressibility which keeps the API stable and enables rapid disintegration and fast dissolution.

In embodiments, the composition comprises GALENIQ 720, which is an agglomerated spherical isomalt of spray-dried sorbitol/mannitol as a co-processed excipient produced from sucrose, with a bulk density of ~0.4, a tapped density of ~0.448, a D50 of ~180 μm. In embodiments, GALENIQ 720 has a pleasant sugar-like, natural taste profile with a sweetening profile almost equal to sucrose and has very low hygroscopic capacity, is chemically extremely stable, and has excellent flow properties.

In embodiments, the composition comprises PEARLITOL SD200, which is a mannitol that is compatible with many pharmaceutical ingredients and is used as a filler and binder, as well as a bulk sweetener, with a bulk density of ~0.48, a tapped density of ~0.58, and a D50 of ~170 μm. In embodiments, PEARLITOL SD200 exhibits a mouthfeel and taste that is slightly sweet (half as sweet as sucrose), has a slight cooling effect in the mouth, and is used to improve the overall mouthfeel of formulations in which it is used, as well as improving the stability in solid and solution states, and is not hygroscopic.

In embodiments, the composition comprises FASTFLO 316, which is a spray-dried mixture of crystalline and amorphous lactose that is compatible with many pharmaceutical ingredients, which is used for improving the compressibility of compositions.

In embodiments, the one or more excipients comprises a texturizer. In embodiments, the texturizer comprises microcrystalline cellulose and/or guar gum. In embodiments, the microcrystalline cellulose and guar gum are present in an approximately a 1:1 ratio, 2:1 ratio, 3:1 ratio, 4:1 ratio, or 5:1 ratio, optionally approximately a 17:3 ratio. In embodiments, the texturizer includes mannitol and microcrystalline cellulose in an approximately a 1:1 ratio, 2:1 ratio, 3:1 ratio, 4:1 ratio, 5:1 ratio, 6:1 ratio, 7:1 ratio, 8:1 ratio, or 9:1 ratio or greater. In embodiments, the one or more excipients comprises a texturizer that includes at least one of AVICEL CE15 or AVICEL HFE 102 (microcrystalline cellulose).

In embodiments, a variety of cellulose-based polymers and cellulose derivatives are included in the composition. For example, in embodiments, hydroxypropyl cellulose ethers (HPC) such as KLUCEL or NISSO HPC, low-substituted hydroxypropyl cellulose ethers (L-HPC), hydroxypropyl methyl cellulose ethers (HPMC) such as SEPPIFIM-LC, PHARMACOAT, METOLOSE SR, METHOCEL-E, OPADRY YS, PRIMAFLO, BENECEL MP824, BENECEL MP843, methylcellulose polymers such as METHOCEL-A, hydroxypropylmethylcellulose acetate stearate AQOAT (HF-LS, HF-LG, HF-MS) and METALOSE Ethylcelluloses (EC) and mixtures thereof such as E461, ETHOCEL, AQUALON-EC, SURLEASE, polyvinyl alcohol (PVA) such as OPADRY AMB, hydroxyethylcelluloses such as NATROSOL, carboxymethylcelluloses and salts of carboxymethylcelluloses (CMC) such as AQUALON-CMC, polyvinyl alcohol and polyethylene glycol co-polymers such as KOLLICOAT monoglycerides (MYVEROL), triglycerides (KLX), polyethylene glycols, modified food starches, acrylic polymers and mixtures of acrylic polymers with cellulose ethers such as EUDRAGIT EPO, EUDRAGI L30D-55, EUDRAGIT FS 30D EUDRAGIT L100-55, EUDRAGIT L100, EUDRAGIT S100, EUDRAGIT RD100, EUDRAGIT E100, EUDRAGIT L12.5, EUDRAGIT S12.5, EUDRAGIT NE30D, and EUDRAGIT NE 40D, cellulose acetate phthalate, SEPIFILMS such as mixtures of HPMC and stearic acid, cyclodextrins, and mixtures of these materials.

In embodiments, the one or more excipients include pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents. In non-limiting embodiments, exemplary carrier materials include, e.g., binders, suspending agents, disintegration agents, filling agents, surfactants, solubilizers, stabilizers, lubricants, wetting agents, diluents, and the like. In embodiments, “pharmaceutically compatible carriers” comprise, but are not limited to, acacia, gelatin, colloidal silicon dioxide, calcium glycerophosphate, calcium lactate, maltodextrin, glycerin, magnesium silicate, polyvinylpyrrolidone (PVP), cholesterol, cholesterol esters, sodium caseinate, soy lecithin, taurocholic acid, phosphatidylcholine, sodium chloride (and other metal-chloride salts), tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is hereby incorporated by reference in their entirety.

In embodiments, the one or more excipients include “plasticizers,” which are compounds used to soften microencapsulation material or film coatings to make them less brittle. In embodiments, exemplary plasticizers include polyethylene glycols such as PEG 300, PEG 400, PEG 600, PEG 1450, PEG 3350, and PEG 800, stearic acid, propylene glycol, oleic acid, triethyl cellulose, and/or triacetin. In embodiments, plasticizers also function as dispersing agents or wetting agents.

In embodiments, the one or more excipients include “solubilizers,” which include compounds such as triethylcitrate, ethyl oleate, ethyl caprylate, sodium lauryl sulfate, sodium docusate, vitamin E (tocofersolan, TPGS), dimethylacetamide, N-methylpyrrolidone, N-hydroxyethylpyrrolidone, hydroxypropyl cyclodextrins, ethanol, n-butanol, isopropyl alcohol, cholesterol, bile salts, polyethylene glycol 200-600, glycofurol, transcutol, and/or dimethyl isosorbide.

In embodiments, the composition and/or the mixed allergen powder comprises starch. In embodiments, the starch is a food starch, such as tapioca starch, potato starch, and/or corn starch. In embodiments, at least a portion of the starch is pregelatinized. In embodiments, the starch comprises approximately 85-95% corn starch and approximately 5-15% pregelatinized starch.

In embodiments, the composition and/or the mixed allergen powder comprises fumed silica. In embodiments, the fumed silica has a bulk density of about or less than about 60 g/L, about or less than about 55 g/L, about or less than about 50 g/L, about or less than about 45 g/L, or about or less than about 40 g/L.

In some embodiments, the mixed allergen powder further comprises fumed silica, preferably fumed silica having a bulk density of about 60 g/L or less.

In embodiments, the composition and/or mixed allergen powder comprises magnesium stearate. In embodiments, magnesium stearate is a simple magnesium salt of a saturated fat called stearic acid, which is a white, water-insoluble powder that provides softness, insolubility in certain solvents, low toxicity, and acts as a “flow agent” to prevent clumping and enables accurate encapsulation.

In embodiments, the overall flowability, cohesivity index, caking strength, and flow stability is determined by one or more of the angle of repose, Carr's Index, FLODEX information, bulk density, tapped density, and Hausner ratio. In embodiments, these measures provide information, which are useful to determine if the composition is suitable for manufacturing in, for example, capsules.

In embodiments, the composition and/or mixed allergen powder has an intrinsic “flowability” quality that is measurable using a FLODEX method, which tests the ability of a powder to fall freely through an aperture in a disk. In embodiments, at least 100 g of the powder composition passes through a 32 mm discharge hole in about 24 seconds, 23 seconds, 22 seconds, 21 seconds, 20 seconds, 19 seconds, 18 seconds, 17 seconds, or 16 seconds.

In embodiments, the composition and/or mixed allergen powder has a cohesivity that is measurable by its angle of repose, which demonstrates the ability of a powder to be piled on a surface. In embodiments, the angle of repose is the angle formed by the horizontal base of a bench surface and the edge of a cone-like pile of powder granules. In embodiments, the composition and/or mixed allergen powder has an average angle of repose less than 45. In embodiments, the average angle of repose is between about 40 degrees and about 45 degrees. In embodiments, the composition and/or mixed allergen powder according to an angle of repose analysis, the composition comprises an average height of cone according to an angle of repose analysis of between about 5 cm to about 7 cm, and/or an average base of cone according to an angle of repose analysis of about 12 cm to about 14 cm.

In embodiments, the composition and/or mixed allergen powder has a powder bridge strength and stability measurable by a Carr's index (e.g., compressibility index). In embodiments, the Carr's Index is measured using the density of the tapped powder minus the density of the bulk powder, divided by the bulk density, and multiplied by 100. In embodiments, the composition or mixed allergen powder has a Carr's Index of about 30 to about 60, or about 35 to about 55, or about 40 to about 50. In embodiments, the composition and/or mixed allergen powder has a Carr's index of less than about 45.

In some embodiments, at least 100 g of the powder passes through a 32 mm discharge hole in about 20 seconds. The powder may have a Carr's index less than 45 and/or an average angle of repose less than 45.

In embodiments, the composition and/or mixed allergen powder has an interparticulate friction that is measurable by a Hausner ratio. In embodiments, the Hausner ratio is measured using a ratio of the tapped powder density to the bulk powder density. In embodiments, the composition comprises a bulk density of about 0.25 g/mL to about 0.35 g/mL; and/or a tapped density of about 0.5 g/mL to about 0.7 g/mL, which results in a Hausner ratio in the range of about 2.8 to about 1.4.

In embodiments, the composition comprises a mixed allergen powder comprising cashew flour, hazelnut flour, walnut flour, and peanut flour, wherein the mixed allergen powder has a particle size of less than about 100 μm and one or more excipients comprising pregelatinized starch, microcrystalline cellulose, guar gum, agglomerated spherical isomalt, mannitol, sorbitol, sucrose, maltodextrin, fumed silica, and magnesium stearate, wherein the one or more excipients has a particle size up to about 150 μm.

In embodiments, the composition consists of (or consists essentially of) a mixed allergen powder consisting of cashew flour, hazelnut flour, walnut flour, and peanut flour, wherein the mixed allergen powder has a particle size of less than about 100 μm, and one or more excipients selected from pregelatinized starch, microcrystalline cellulose, guar gum, agglomerated spherical isomalt, mannitol, fumed silica, and magnesium stearate, wherein the one or more excipients has a particle size up to about 150 μm.

In embodiments, the composition comprises (or consists essentially of) the formulation, as substantially outlined in Table D, including cashew flour at about 1.63% (w/w), hazelnut flour at about 1.38% (w/w), walnut flour at about 1.11% (w/w), peanut flour at about 1.02% (w/w), STARCAP 1500 (corn starch and pregelatinized starch) at about 10% (w/w), AVICEL PH-101 (partially depolymerized microcrystalline alpha-cellulose) at about 83.85% (w/w), CAB-O-SIL M-5P (fumed silica) at about 0.5% (w/w), and magnesium stearate at about 0.5% (w/w).

In embodiments, the composition comprises (or consists essentially of) the formulation, as substantially outlined in Table H, including cashew flour at about 20.05% (w/w), hazelnut flour at about 17.03% (w/w), walnut flour at about 13.70% (w/w), peanut flour at about 12.61% (w/w), STARCAP 1500 (corn starch and pregelatinized starch) at about 10% (w/w), AVICEL PH-101 (partially depolymerized microcrystalline alpha-cellulose) at about 25.61% (w/w), CAB-O-SIL M-5P (fumed silica) at about 0.5% (w/w), and magnesium stearate at about 0.5% (w/w).

In embodiments, the composition comprises (or consists essentially of) the formulation, as substantially outlined in Table L, including cashew flour at about 20.05% (w/w), hazelnut flour at about 17.03% (w/w), walnut flour at about 13.70% (w/w), peanut flour at about 12.61% (w/w), STARCAP 1500 (corn starch and pregelatinized starch) at about 10% (w/w), PARTEK SI 150 (sorbitol) at about 10% (w/w), PARTEK M200 (spray-dried mannitol) at about 14.11% (w/w), CAB-O-SIL M-5P (fumed silica) at about 2% (w/w), and magnesium stearate at about 0.5% (w/w).

In embodiments, the composition comprises (or consists essentially of) the formulation, as substantially outlined in Table P, including cashew flour at about 20.05% (w/w), hazelnut flour at about 17.03% (w/w), walnut flour at about 13.70% (w/w), peanut flour at about 12.61% (w/w), STARCAP 1500 (corn starch and pregelatinized starch) at about 34.11% (w/w), CAB-O-SIL M-5P (fumed silica) at about 2% (w/w), and magnesium stearate at about 0.5% (w/w).

In embodiments, the composition comprises (or consists essentially of) the formulation, as substantially outlined in Table T, including cashew flour at about 20.05% (w/w), hazelnut flour at about 17.03% (w/w), walnut flour at about 13.70% (w/w), peanut flour at about 12.61% (w/w), GALENIQ 720 (agglomerated spherical isomalt of spray-dried sorbitol/mannitol) at about 34.11% (w/w), CAB-O-SIL M-5P (fumed silica) at about 2% (w/w), and magnesium stearate at about 0.5% (w/w).

In embodiments, the composition comprises (or consists essentially of) the formulation, as substantially outlined in Table X, including cashew flour at about 20.05% (w/w), hazelnut flour at about 17.03% (w/w), walnut flour at about 13.70% (w/w), peanut flour at about 12.61% (w/w), PARTEK M200 (spray-dried mannitol) at about 34.11% (w/w), CAB-O-SIL M-5P (fumed silica) at about 2% (w/w), and magnesium stearate at about 0.5% (w/w).

In embodiments, the composition comprises (or consists essentially of) the formulation, as substantially outlined in Table BB, including cashew flour at about 20.05% (w/w), hazelnut flour at about 17.03% (w/w), walnut flour at about 13.70% (w/w), peanut flour at about 12.61% (w/w), DI-PAC (granulated sugar, maltodextrin) at about 34.11% (w/w), CAB-O-SIL M-5P (fumed silica) at about 2% (w/w), and magnesium stearate at about 0.5% (w/w).

In embodiments, the composition comprises (or consists essentially of) the formulation, as substantially outlined in Table FF, including cashew flour at about 10.55% (w/w), hazelnut flour at about 8.96% (w/w), walnut flour at about 7.20% (w/w), peanut flour at about 6.63% (w/w), STARCAP 1500 (corn starch and pregelatinized starch) at about 20% (w/w), PARTEK SI 150 (sorbitol) at about 20% (w/w), PARTEK M200 (spray-dried mannitol) at about 24.16% (w/w), CAB-O-SIL M-5P (fumed silica) at about 2% (w/w), and magnesium stearate at about 0.5% (w/w).

In embodiments, the composition comprises (or consists essentially of) the formulation, as substantially outlined in Table JJ, including cashew flour at about 20.05% (w/w), hazelnut flour at about 17.03% (w/w), walnut flour at about 13.70% (w/w), peanut flour at about 12.61% (w/w), STARCAP 1500 (corn starch and pregelatinized starch) at about 9% (w/w), PARTEK SI 150 (sorbitol) at about 9% (w/w), PARTEK M200 (spray-dried mannitol) at about 12.61% (w/w), micronized talc at about 5% (w/w), CAB-O-SIL M-5P (fumed silica) at about 0.5% (w/w), and magnesium stearate at about 0.5% (w/w).

In embodiments, the composition comprises (or consists essentially of) the formulation, as substantially outlined in Table NN, including cashew flour at about 20.05% (w/w), hazelnut flour at about 17.03% (w/w), walnut flour at about 13.70% (w/w), peanut flour at about 12.61% (w/w), STARCAP 1500 (corn starch and pregelatinized starch) at about 10% (w/w), PARTEK SI 150 (sorbitol) at about 10% (w/w), PEARLITOL SD200 (mannitol) at about 14.11% (w/w), CAB-O-SIL M-5P (fumed silica) at about 2% (w/w), and magnesium stearate at about 0.5% (w/w).

In embodiments, the composition comprises (or consists essentially of) the formulation, as substantially outlined in Table RR, including cashew flour at about 20.05% (w/w), hazelnut flour at about 17.03% (w/w), walnut flour at about 13.70% (w/w), peanut flour at about 12.61% (w/w), STARCAP 1500 (corn starch and pregelatinized starch) at about 10% (w/w), PEARLITOL SD200 (mannitol) at about 24.11% (w/w), CAB-O-SIL M-5P (fumed silica) at about 2% (w/w), and magnesium stearate at about 0.5% (w/w).

In embodiments, the composition comprises (or consists essentially of) the formulation, as substantially outlined in Table W, including cashew flour at about 20.05% (w/w), hazelnut flour at about 17.03% (w/w), walnut flour at about 13.70% (w/w), peanut flour at about 12.61% (w/w), STARCAP 1500 (corn starch and pregelatinized starch) at about 10% (w/w), FAST FLO 316 (lactose) at about 24.11% (w/w), CAB-O-SIL M-5P (fumed silica) at about 2% (w/w), and magnesium stearate at about 0.5% (w/w).

In embodiments, the composition comprises (or consists essentially of) the formulation, as substantially outlined in Table ZZ, including cashew flour at about 20.37% (w/w), hazelnut flour at about 17.29% (w/w), walnut flour at about 13.91% (w/w), peanut flour at about 12.81% (w/w), STARCAP 1500 (corn starch and pregelatinized starch) at about 10.42% (w/w), GALENIQ 720 (agglomerated spherical isomalt of spray-dried sorbitol/mannitol) at about 11.25% (w/w), PEARLITOL SD200 (mannitol) at about 3.13% (w/w), AVICEL HFE-102 (microcrystalline cellulose and mannitol) at about 8.33% (w/w), CAB-O-SIL M-5P (fumed silica) at about 2% (w/w), and magnesium stearate at about 0.5% (w/w).

In embodiments, the composition comprises (or consists essentially of) the formulation, as substantially outlined in Table DDD, including cashew flour at about 20.37% (w/w), hazelnut flour at about 17.29% (w/w), walnut flour at about 13.91% (w/w), peanut flour at about 12.81% (w/w), STARCAP 1500 (corn starch and pregelatinized starch) at about 10.42% (w/w), GALENIQ 720 (agglomerated spherical isomalt of spray-dried sorbitol/mannitol) at about 11.25% (w/w), PEARLITOL SD200 (mannitol) at about 3.13% (w/w), AVICEL CE-15 (microcrystalline cellulose and guar gum) at about 8.33% (w/w), CAB-O-SIL M-5P (fumed silica) at about 2% (w/w), and magnesium stearate at about 0.5% (w/w).

In embodiments, the composition comprises (or consists essentially of) the formulation, as substantially outlined in Table HHH, including cashew flour at about 20.37% (w/w), hazelnut flour at about 17.29% (w/w), walnut flour at about 13.91% (w/w), peanut flour at about 12.81% (w/w), STARCAP 1500 (corn starch and pregelatinized starch) at about 14.58% (w/w), GALENIQ 720 (agglomerated spherical isomalt of spray-dried sorbitol/mannitol) at about 14.37% (w/w), PEARLITOL SD200 (mannitol) at about 4.17% (w/w), CAB-O-SIL M-5P (fumed silica) at about 2% (w/w), and magnesium stearate at about 0.5% (w/w).

In embodiments, the composition comprises (or consists essentially of) the formulation, as substantially outlined in Table 4, including cashew flour at about 20.47% (w/w), hazelnut flour at about 17.38% (w/w), walnut flour at about 13.98% (w/w), peanut flour at about 12.87% (w/w), STARCAP 1500 (corn starch and pregelatinized starch) at about 10.21% (w/w), GALENIQ 720 (agglomerated spherical isomalt of spray-dried sorbitol/mannitol) at about 18.89% (w/w), PEARLITOL SD200 (mannitol) at about 4.19% (w/w), and CAB-O-SIL M-5P (fumed silica) at about 2.01% (w/w).

In embodiments, the composition comprises (or consists essentially of) the formulation, as substantially outlined in Table 4, including cashew flour at about 20.37% (w/w), hazelnut flour at about 17.29% (w/w), walnut flour at about 13.91% (w/w), peanut flour at about 12.81% (w/w), STARCAP 1500 (corn starch and pregelatinized starch) at about 10.16% (w/w), GALENIQ 720 (agglomerated spherical isomalt of spray-dried sorbitol/mannitol) at about 18.80% (w/w), PEARLITOL SD200 (mannitol) at about 4.17% (w/w), CAB-O-SIL M-5P (fumed silica) at about 2% (w/w), and magnesium stearate at about 0.5% (w/w).

In embodiments, the composition comprises (or consists essentially of) the formulation, as substantially outlined in Table 4, including cashew flour at about 10.56% (w/w), hazelnut flour at about 9.97% (w/w), walnut flour at about 3.98% (w/w), peanut flour at about 3.81% (w/w), STARCAP 1500 (corn starch and pregelatinized starch) at about 8.97% (w/w), GALENIQ 720 (agglomerated spherical isomalt of spray-dried sorbitol/mannitol) at about 25.92% (w/w), PEARLITOL SD200 (mannitol) at about 8.85% (w/w), and CAB-O-SIL M-5P (fumed silica) at about 1.94% (w/w).

In embodiments, the composition comprises (or consists essentially of) the formulation, as substantially outlined in Table 4, including cashew flour at about 1.63% (w/w), hazelnut flour at about 1.38% (w/w), walnut flour at about 1.11% (w/w), peanut flour at about 1.02% (w/w), STARCAP 1500 (corn starch and pregelatinized starch) at about 15% (w/w), GALENIQ 720 (agglomerated spherical isomalt of spray-dried sorbitol/mannitol) at about 67.35% (w/w), PEARLITOL SD200 (mannitol) at about 10% (w/w), CAB-O-SIL M-5P (fumed silica) at about 2% (w/w), and magnesium stearate at about 0.5% (w/w).

In embodiments, the composition is a “pharmaceutical composition,” where the composition is specifically formulated to be suitable for oral administration (e.g., a capsule and/or sachet) to be ingested by a subject for preventing and/or treating a nut allergy. In embodiments, any features that apply to the “composition” apply to the “pharmaceutical composition.”

### Methods of Manufacturing Mixed Nut Allergen Powders and Compositions Thereof

Another aspect of the present disclosure is a method of making a mixed allergen powder, the method comprising: mixing a plurality of tree nut flours with an excipient. Yet another aspect of the present disclosure is a method of making a mixed allergen powder, the method comprising: providing an excipient that has been spray-dried; and mixing a plurality of tree nut flours with the excipient. In each of these methods, the excipient has a particle size up to about 150 microns, for example, no greater than about 140 microns, no greater than about 130 microns, no greater than about 120 microns, no greater than about 110 microns, or no greater than about 100 microns. Each of the tree nut flours may have a particle size up to about 100 microns, for example, no greater than about 75 microns, no greater than about 50 microns, or no greater than about 40 microns.

In aspects, the present disclosure is directed to a method of making a composition comprising a mixed allergen powder. In embodiments the method comprises mixing one or more nut flours with one or more excipients. In embodiments, the one or more nut flours each have been processed to have a particle size of less than about 100 μm, and the one or more excipients each have been processed to have a particle size of less than about 150 μm.

In embodiments, the processing of the nut flours and/or excipients to achieve the desired particle size includes pharmacological techniques, such as dry mixing, direct compression, milling, dry or non-aqueous granulation, wet granulation, or fusion. In embodiments, other methods include, e.g., spray drying, pan coating, melt granulation, granulation, fluidized bed spray drying or coating (e.g., Wurster coating), tangential coating, top spraying, tableting, extruding and the like. Persons skilled in the art, upon benefit of this disclosure in its entirety, will be aware of the various pharmacological techniques useful for obtaining the desired particles sizes of nut flours and excipients.

In embodiments, the method comprises providing one or more excipients that have been spray-dried, and mixing one or more nut flours with the excipient(s). In embodiments, the one or more excipients have a particle size up to about 150 microns, for example, no greater than about 140 microns, no greater than about 130 microns, no greater than about 120 microns, no greater than about 110 microns, or no greater than about 100 microns. In embodiments, each of the nut flours has a particle size up to about 100 microns, for example, no greater than about 75 microns, no greater than about 50 microns, or no greater than about 40 microns.

Without wishing to be bound by theory, the small, controlled particle sizes are conducive to initiating controlled immunological responses for orally ingested allergen desensitization, while having physical properties to be manufactured at-scale into oral formulations. Without wishing to be bound by theory, because the mass amounts of the nut allergens are small (e.g., about 0.5 mg+), the physical attributes of the formulations, as demonstrated in the Examples herein, are conducive to uniform dosages between batches of individual capsules or sachets.

In embodiments, the one or more nut flours comprises cashew flour, hazelnut flour, walnut flour, and peanut flour; alternatively the one or more nut flours consist of cashew flour, hazelnut flour, walnut flour, and peanut flour.

In some embodiments, the method comprises defatting at least one of the plurality of tree nut flours to form a cake, preferably having a fat content of less than 12 wt. %, before mixing the plurality of tree nut flours with the excipient. The method may further comprise milling the cake to a predetermined particle size before mixing the plurality of tree nut flours with the excipient. The milling preferably forms a uniform, free-flowing blend of the plurality of tree nut flours. In some embodiments, the method further comprises filling the mixed allergen powder into capsules.

In embodiments, the method comprises defatting one or more nut flours to form one or more nut flour cakes. In embodiments, the one or more nut flours and/or one of more nut flour cakes has been defatted to reduce the fat content to less than about 10 wt. % (10% w/w), 12 wt. % (12% w/w), 15 wt. % (15% w/w), or more before mixing the one or more nut flours with the one or more excipients. Defatting of nut flour, in embodiments, is achieved using various methods, such as using compression (e.g., using a press to expel oils from the nut flour), and/or using an absorbent layer (e.g., paper towel/fiber or cheesecloth) to filter/absorb oils from the nut flour, and/or using solvents (e.g., water, alcohols, etc.) to extract/dissolve oils from the nut flour.

In embodiments, the method includes milling the one or more nut flour cakes to a predetermined particle size before mixing the one or more nut flours with the one or more excipients. In embodiments, the milling preferably forms a uniform, free-flowing blend of the one or more nut flours and/or nut flour cakes. In embodiments, the milling is performed to produce a particle size is in the range of about 100 μm or less (e.g., about 90 μm, 80 μm, 70 μm, 60 μm, 50 μm, 40 μm, or less). In embodiments, the milling of the one or more nut flours occurs prior to admixing with one or more excipients, and/or during admixing with one or more excipients, and/or after admixing with one or more excipients.

In embodiments, the method comprises passing or co-sieving the one or more nut flours and/or the one or more excipients through a mesh screen/sieve. In embodiments, the mixing includes blending the materials by discharging through one or more difference sizes of a mesh screen/sieve. In embodiments, the mesh screen/sieve has a U.S. mesh number of about 10 (e.g., ~2000 μm pore size) to about 400 (e.g., ~37 μm pore size).

In embodiments, the method comprises performing one or more of analyses to determine the overall flowability, cohesivity index, caking strength, and flow stability of the composition. In embodiments, the method comprises performing one or more of an angle of repose analysis, Carr's Index analysis, FLODEX analysis (e.g., FLOWDEX analysis), determining a bulk density and/or tapped density and/or Hausner ratio. In embodiments, the method comprises performing one or more of these analyses prior to formulating into a capsule or sachet.

In embodiments, the method comprises performing one or more analyses to determine and/or control the concentration of allergen peptides. In embodiments, the method comprises performing one or more HPLC steps (e.g., chromatography to determine retention volumes relative to a protein standard), one or more steps of an enzyme-linked immunosorbent assay (ELISA) (e.g., using antibodies which bind specific proteins/allergens to determine their concentration or presence), one or more steps of a gel electrophoresis analyses (e.g., SDS-PAGE, native or non-reducing gel electrophoresis), one or more steps of Western blotting and/or immunoprecipitation (e.g., a pulldown assay or immunoblotting to determine the presence or concentration of allergen proteins/peptides), and/or one or more steps of a liquid chromatography mass spectroscopy (LC-MS/MS) analysis. Persons skilled in the art will recognize the various techniques useful for protein/peptide concentration determination.

In embodiments, the protein content of nut flours comprise a purity factor, for example, of about or at least about 0.3 to about or at least about 0.5. In embodiments, the total protein content of the one or more nut flours ranges from about 20% to about 50% total protein content. In embodiments, the total protein content of the composition ranges from about 1.5% (w/w) (e.g., in a ~0.8 mg capsule) to about 35% (w/w) (e.g., in a ~1200 mg capsule).

In embodiments, the method further comprises formulating the mixed allergen powder into a form suitable for oral administration. This includes, in embodiments, filling the composition comprising the mixed allergen powder into capsules, or forming the powder into sachets.

### Methods of Use to Prevent or Treat Nut Allergy

Another aspect of the present disclosure is a method of preventing or treating at least one tree nut allergy in a subject, preferably by oral immunotherapy, the method comprising administering any mixed allergen powder disclosed herein and/or a mixed allergen powder made by any method disclosed herein to the subject.

The compositions described herein can be formulated for administration to a subject by any conventional means including, but not limited to, oral administration routes. As used herein, the term “subject” is used to mean an animal, preferably a mammal, including a human or non-human. The terms subject and subject may be used interchangeably. The formulations are for prevention and treatment of symptoms associated with exposure to limited amounts of peanut allergen in children and adults. In some embodiments, a subject is from about 4 to about 26 years of age.

In aspects, the present disclosure provides a method or use of preventing or treating one or more nut allergy in a subject in need thereof by administering one or more composition(s), as described herein. In embodiments, the method or use includes an oral immunotherapy (OIT) treatment, where one or more doses of the composition, which comprises one or more nut allergen peptides/proteins, are consumed by the subject to prevent or treat the associated nut allergy.

In embodiments, the subject has one or more of a cashew allergy, hazelnut allergy, walnut allergy, and peanut allergy. In embodiments, a nut allergy is detectable by analyzing a sample from a subject (e.g., a blood or sera sample) for the presence of nut-specific IgE antibodies (e.g., cashew-specific IgEs, hazelnut-specific IgEs, walnut-specific IgEs, and peanut-specific lgEs). In embodiments, the presents of nut-specific IgEs is detectable using an antibody-binding assay and/or an ELISA-based assay with a sample taken from a subject (e.g., using nut allergen proteins to detect antibodies that bind them). In embodiments, the method includes providing a subject a composition of mixed nut allergen powder, where the powder contains both allergenic peptides and non-allergenic peptides, in relation to the subject's allergies (e.g., where the subject has a peanut allergy and an allergy to at least one of the tree nuts, but not each of cashew, hazelnut, and walnut allergies). In embodiments, the method includes determining or receiving a level of nut-specific IgE levels to select a subject for treatment.

In embodiments, the method or use results in a decrease in nut-specific IgE levels in the subject over time. In embodiments, the level of nut-specific IgEs temporarily increases (e.g., over the course of weeks or months), and then begins to decrease over time (e.g., over the course of or months or years). In embodiments, the level of nut-specific IgEs is compared to a threshold level to determine one or more method steps in relation to the oral immunotherapy (OIT). For example, in embodiments, a level of 100 KU/L (e.g., 100 IU/mL) of one or more of cashew-specific IgEs, hazelnut-specific IgEs, walnut-specific IgEs, and/or peanut-specific IgEs is used to temporarily halt or delay one or more doses, reduce the amount of one or more doses, or stop treatment altogether.

In embodiments, the method comprises administering one or more doses of the composition comprising the mixed nut allergen powder, receiving a measurement of nut-specific IgE levels in the subject (e.g., including measuring the nut-specific IgE levels), comparing the level of nut-specific IgEs to a threshold level of nut-specific IgEs, and adjusting a subsequent dose of the composition based on the comparison. In embodiments, the threshold level is about or at least about 70 kU/L, or about or at least about 100 kU/L, of one or more of cashew-specific IgEs, hazelnut-specific IgEs, walnut-specific IgEs, and peanut-specific IgEs.

In embodiments, the OIT comprises one or more phases of dosing, for example, an initial escalation phase, an up-dosing phase, and/or a maintenance phase. In embodiments, the initial escalation phase comprises one or more doses provided within about a 24-hour period (e.g., on day 1 of OIT), where the doses comprises about or less than about 0.5 mg, 1.0 mg, 1.5 mg, 2.0 mg, 2.5 mg, 3.0 mg, or 6.0 mg of nut protein and/or nut flour. In embodiments, the up-dosing phase comprises one or more doses provided daily over a course of about 1 week, 2 weeks, 3 weeks, 1 month, 2 months, 3 months, 6 months, or about 1 year or more, where the dose increases over predetermined time periods (e.g., a dose increase every week, every 2 weeks, every 3 weeks, every 4 weeks, etc.). In embodiments, the one or more doses of the up-dosing phase comprise about or less than about 3 mg, 6 mg, 12 mg, 20 mg, 80 mg, 120 mg, 160 mg, 200 mg, 240 mg, or 300 mg of nut protein and/or nut flour. In embodiments, the maintenance phase comprises one or more doses provided daily over the course of 1 week, 2 weeks, 3 weeks, 1 month, 2 months, 3 months, 6 months, or about 1 year or more, where the phase has periods of constant dose and/or dose increases over predetermined time periods (e.g., a dose increase every 2 weeks, every month, etc.). In embodiments, the one or more doses of the maintenance phase comprise about or less than about 300 mg, 600 mg, or 1000 mg or more of nut protein and/or nut flour.

In embodiments, one or more doses of the mixed nut allergen composition herein are administered as an oral capsule. In embodiments, one or more doses of the mixed nut allergen composition herein are administered as a powder sachet which is admixed with an edible composition (e.g., food or beverage).

In embodiments, the subject is a human (e.g., a child, adolescent, teenager, adult, or geriatric). In embodiments, the subject is about 4 years old and about 11 years old, or about 4 years old to about 17 years old. In embodiments, the subject is about 12 years of age or older, or about 18 years of age or older.

In embodiments, a subject is from about 4 to about 26 years of age. In embodiments, the age the subject is obtained, and assessment of the risk of a systemic allergic response is further based on the age of the subject, wherein an age between 12 years of age and 17 years of age indicates an increased risk of a systemic allergic response compared to an age between about 4 years old and about 11 years old, and/or the dose is adjusted accordingly.

The compositions described herein can be formulated for administration to a subject by any conventional means including, but not limited to, oral administration routes. As used herein, the term “subject” is used to mean an animal, preferably a mammal, including a human or non-human. The terms subject and patient may be used interchangeably. The formulations are for prevention and treatment of symptoms associated with exposure to limited amounts of peanut allergen in children and adults.

### Kits

In aspects, the present disclosure provides kits and systems including one of more compositions, pharmaceutical compositions, and/or doses thereof, e.g., for performing methods described herein.

In embodiments, the kit comprises one or more dosage containers comprising one or more detached single dose packages comprising the equivalent dosages for an oral immunotherapy (OIT) schedule. In embodiments, the single dose packages in different dosage containers comprise different dosages of the OIT schedule. In embodiments, the dosage containers comprises a dosage identifier that associates the dosage container with the dosage of OIT composition contained by the single dose packages contained in the dosage container. In embodiments, the kit comprises a housing having an internal cavity that retains the plurality of dosage containers. In embodiments, the kit additionally comprises instructions for administering the composition in accordance with one or more dosing schedules, as described herein.

### EXAMPLES

The present disclosure is understood by reference to the following non-limiting examples, which are provided as exemplary embodiments. The following examples are presented to more fully illustrate embodiments and should not be construed as limiting the scope of the present disclosure. While certain embodiments have been shown and described herein, such embodiments are provided by way of example only. Numerous variations, changes, and substitutions may occur to those skilled in the art without departing from the spirit and scope of the disclosure. It should be understood that various alternatives to the embodiments described herein are employable in practicing the methods described herein.

### Example 1: Multi-Nut Formulation Detection Method

This Example describes an overview of the liquid chromatography-mass spectrometry/mass spectrometry (LC-MS/MS) method used for the detection of marker peptides in cashew, hazelnut, peanut and walnut in multi-nut formulations ranging from 0.8-1200 mg dosage strength.

Equal weights of the roasted and partially defatted Duyvis flour samples of cashew, hazelnut, walnut, and peanut were mixed in a tumbler for 8 hours to prepare the primary flour standard. Peptide standards for each nut were synthesized (Thermo Pierce) at greater than 95% purity, and are shown in Table 1. The isotopically labeled internal standards were AQUA basic (95% purity) where the c-terminal amino acid (either R or K) was substituted for an isotopically labeled R (+10 daltons) or K (+8 daltons).

TABLE 1
Mixed Allergen Peptides.

| Allergen | Amino acid Sequence | |
|---|---|---|
| Protein | (internal standards are bolded) | Modification(s) |
| Ana o 2-292 | ADIYTPEVGR [SEQ ID NO: 1] | — |
| Ana o 2-350 | GQVQVVDNFGNR [SEQ ID NO: 2] | — |
| Ana o 3-042 | QFEEQQR [SEQ ID NO: 3] | — |
| Ana o 3-112 | ELYETASELPR [SEQ ID NO: 4] | — |
| Cor a 9-043 | LNALEPTNR [SEQ ID NO: 5] | — |
| Cor a 9-341 | ADIYTEQVGR [SEQ ID NO: 6] | — |
| Cor a 11-092 | LLSGIENFR [SEQ ID NO: 7] | — |
| Cor a 11-395 | GNIVNEFER [SEQ ID NO: 8] | — |
| Jug r 2-193 | HESEEGEVK [SEQ ID NO: 9] | — |
| Jug r 2-343 | FFDQQEQR [SEQ ID NO: 10] | — |
| Jug r 4-041 | LDALEPTNR [SEQ ID NO: 11] | — |
| Jug r 4-336 | ADIYTEEAGR [SEQ ID NO: 12] | — |
| Ara h 1-128 | RPSHQQPR [SEQ ID NO: 13] | — |
| Ara h 1-541 | IFLAGDKDNVIDQIEK [SEQ ID NO: 14] | — |
| Ara h 2-023 | QQWELQGDR [SEQ ID NO: 15] | — |
| Ara h 2-160 | CDLEVESGGR [SEQ ID NO: 16] | c: Carbamidomethylation |

Peptides were dissolved in water or mixtures of water and acetonitrile to make 1 mg/ml solutions. The 16 peptides were mixed together to make a 50 μg/mL mixture, which was diluted serially to make standards of 30, 10, 3, 1, 0.3, 0.1, 0.03 and 0.01 μg/mL for generating a calibration curve.

The primary multi-nut flour standard was extracted and digested at a level of 20 mg/mL. After the last step in the digestion, the supernatant was taken and diluted by 2× to make a calibration curve of 20, 10, 5, 2.5, 1.25, 0.625, 0.3125, 0.156225 and 0.078125 mg/mL of nut flour. The calibration levels were adjusted from weight to specific nut protein levels based on the percent protein content shown in Table 2.

TABLE 2
Nut protein levels for multi-nut calibration
standards.

| API | % Protein content |
|---|---|
| Cashew flour | 0.32 |
| Hazelnut flour | 0.38 |
| Walnut flour | 0.47 |
| Peanut Flour | 0.50 |

Multi-nut mixtures were extracted, digested, and analyzed by LC-MS/MS using an AGILENT Model G6470A Triple Quadrupole mass spectrometer. The instrument was calibrated over the m/z range using the AGILENT tuning solution (AGILENT part #G1969-85000). The electrospray source conditions were as follows: capillary 4000 V, nozzle 1000 V, fragmentor 160 V, drying gas 350° C. at 8.5 L/min, nebulization gas 40 psi and sheath gas 350° C. at 12 L/min. The first 2.25 min of the run was diverted to waste. AGILENT MASSHUNTER mass spectroscopy software was used to acquire and process the data (acquisition version B.10.00 and qualitative and quantitative data analysis version B.10.00).

Quantitation for all samples analyzed was based on the ratio of peak areas for the unlabeled peptide to labeled peptide vs. concentration of the unlabeled peptide to generate a calibration curve. Calibration fits were linear with 1/x weighing. Using the calibration curve, quality controls and samples could be quantified based on the ratio of peak areas for the unlabeled peptide to labeled peptide. The amount of peptide generated from the multi-nut flour standard is then used to generate a calibration of peptide concentration vs protein concentration with linear fit.

### Example 2: Characterization of Multi-Nut Formulations

This Example provides the results of the characterization of various multi-nut formulations. A total of eighteen different multi-nut formulations were produced and characterized.

### A. Multi-Nut Formulation Flowability

Each multi-nut formulation was analyzed for density, average angle of repose, FLOWDEX (powder flowability analysis), and Carr's index. Angle of repose represents the steepest angle of descent relative to a horizontal plane in which the powder is poured to create an angle from 0 degrees to 90 degrees, wherein a lower angle indicates a better flow. The Carr's index is an indication of the compressibility of a powder. Finally, the FLOWDEX powder flowability analysis evaluated the ability of the formulation to flow through a particular pore width. The flowability was determined as a combination of angle of repose, Carr's index, and FLOWDEX information.

i. PALFORZIA

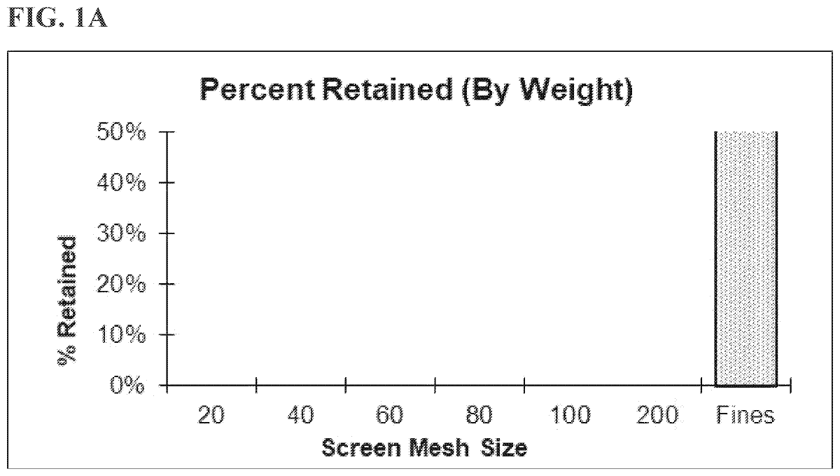

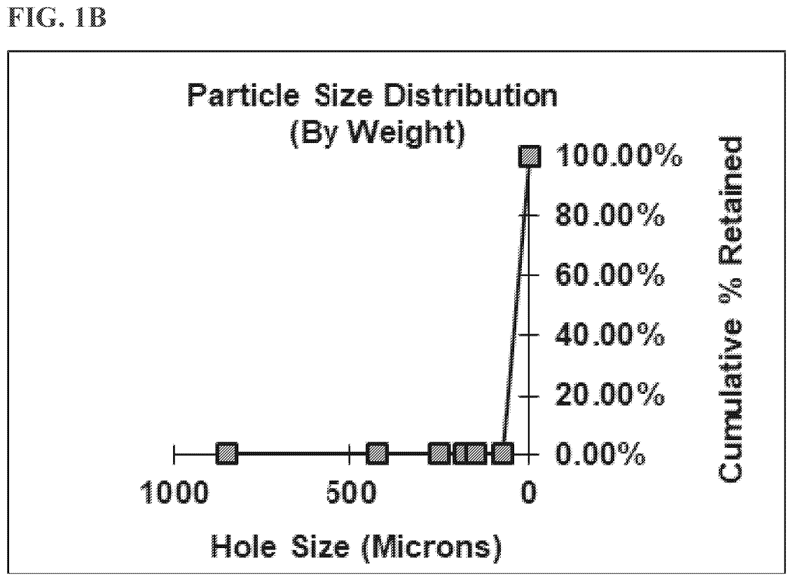

The first formula tested was PALFORZIA which is a peanut powder-based OIT. Results of the sieve analysis of 100 mg of PALFORZIA are shown in FIGS. 1A-1B. Table A below shows the results of the density analysis of PALFORZIA (100 mg).

TABLE A
PALFORZIA density analysis

| | | | |
|---|---|---|---|
| Material weight (g) | 31.091 | Material weight (g) | 31.09 |
| Number of taps | 2500 | Bulk density (g/mL) | 0.32 |
| Bulk volume (mL) | 96 | Tapped density (g/mL) | 0.58 |
| Tapped Volume (mL) | 54 | Carr's index | 43.75 |

The density analysis showed that PALFORZIA has a Carr's index of 43.75. The angle of repose analysis of PALFORZIA is shown in Table B.

TABLE B
PALFORZIA angle of repose analysis.

| | Height of | Base of | Angle of |
|---|---|---|---|
| Test | Cone (cm) | Cone (cm) | Repose |
| 1 | 5.8 | 11.9 | 44.3 |
| 2 | 5.9 | 12.4 | 43.6 |
| 3 | 6.3 | 12.3 | 45.7 |

| | |
|---|---|
| Average | 44.5 |
| % RSD | 2.4 |

The FLOWDEX results indicated that PALFORZIA did not pass through a 34 mm opening.

ii. 21003-1

The nut flour composition of formulation 21003-1 in a 200 g batch size is shown in Table C.

TABLE C
21003-1 nut flour composition.

| Active Pharmaceutical | Protein content | | Purity |
|---|---|---|---|
| Ingredient (API) | (HPLC) | Water | factor |
| Cashew flour | 32 | 4.1 | 0.30688 |
| Hazelnut flour | 38 | 4.9 | 0.36138 |
| Walnut flour | 47 | 4.4 | 0.44932 |
| Peanut flour | 50 | 2.4 | 0.488 |

Formulation 21003-1 was prepared with STARCAP 1500, AVICEL PH-101, CAB-O-SIL® M-5P, and magnesium stearate.

STARCAP 1500 is a co-processed mixture of globally accepted excipients, corn starch, and pregelatinized starch, with a bulk density of 0.42, a tapped density of 0.58, a median particle diameter (D50) of ~90 μm. The shape and size of STARCAP 1500 results in improved flow over similarly sized microcrystalline cellulose (MCC). Moreover, STARCAP 1500 is inert, may improve stability, provides weight uniformity due to its excellent flow in high speed capsule filling operation, and starts to dissolve is water by acting as a binder, thus aiding in mouthfeel upon ingestion.

AVICEL PH 101 is a purified, partially depolymerized microcrystalline alpha-cellulose made by acid hydrolysis of specialty wood pulp, with a D50 of ~50.

CAB-O-SIL M-5P is a fumed silica that is compatible with many pharmaceutical ingredients, and has a particle surface area of 200 m²/g and a bulk density of <60 g/L. It adheres readily to hydrophilic ingredients, functioning as an excellent glidant, and is easy to disperse.

Magnesium stearate is a simple salt made up of two substances, a saturated fat called stearic acid and the mineral magnesium. Magnesium stearate is a white, water-insoluble powder. Its applications exploit its softness, insolubility in many solvents, and low toxicity.

To generate the 21003-1 formulation, 103.01 g of APIs and 10.00 g of CAB-O-SIL M-5P were bag blended. The resulting blend was milled through a 24R CNC Router and a PHILIPS TWIST HR-1701. A ¼ geometric blend with a 500 g batch size was then produced by mixing 168.50 g AVICEL PH-101 (168.50 g blender charge), 50 g STARCAP 1500 (218.50 g blender charge), 113.01 g of the blended API+CAB-O-SIL M-5P mixture (331.50 g blender charge), and another 168.50 g AVICEL PH-101 (500.00 g blender charge), in that order. The resulting ¼ geometric blend was milled through a 32R CNC Router and a PHILIPS TWIST HR-1701. The other excipients were then adjusted to the yield. A main blend was then produced by mixing 670.00 g AVICEL PH-101 (670.00 g blender charge), 150.00 g STARCAP 1500 (820.00 g blender charge), 500.00 g of the ¼ geometric blend (1320.00 g blender charge), and another 670.00 g AVICEL PH-101 (1990.00 g blender charge), in that order. The resulting main blend was milled through a 32R CNC Router and a PHILIPS TWIST HR-1701. The main blend was lubricated, thus resulting in the final main blend #2 21003-1 formulation.

A summary of the composition of a 0.8 mg dosage of formulation 21003-1 is shown in Table D.

TABLE D
Formulation 21003-1 composition.

| | 0.8 mg Dosage 21003-1 | | | |
|---|---|---|---|---|
| | | | Adjusted | Adjusted |
| Ingredient | % w/w | mg/dose | g/batch | °/ow/w |
| Cashew flour (Lot: | 0.50 | 0.20 | 32.59 | 1.63 |
| 21002-17) | | | | |
| Hazelnut flour (Lot: | 0.50 | 0.20 | 27.67 | 1.38 |
| 21002-20) | | | | |
| Walnut flour (Lot: | 0.50 | 0.20 | 22.26 | 1.11 |
| 21002-23) | | | | |
| Peanut Flour (Lot: | 0.50 | 0.20 | 20.49 | 1.02 |
| 121FA04020) | | | | |
| STARCAP 1500 | 10.00 | 4.00 | 200.00 | 10.00 |
| AVICEL PH-101 | 87.00 | 34.80 | 1676.99 | 83.85 |
| CAB-O-SIL M-5P | 0.50 | 0.20 | 10.00 | 0.50 |
| Magnesium stearate | 0.50 | 0.20 | 10.00 | 0.50 |
| | 100.00 | 40.00 | 2000.00 | 100.00 |

Table E below shows the results of the density analysis of formulation 21003-1.

TABLE E
Formulation 21003-1 density analysis.

| | | | |
|---|---|---|---|
| Material weight (g) | 88.99 | Material weight (g) | 88.99 |
| Number of taps | 2500 | Bulk density (g/mL) | 0.36 |
| Bulk volume (mL) | 246 | Tapped density (g/mL) | 0.58 |
| Tapped Volume (mL) | 154 | Carr's index | 37.40 |

The density analysis showed that formulation 21003-1 has a Carr's index of 37.40. The angle of repose analysis of formulation 21003-1 is shown in Table F.

TABLE F
Formulation 21003-1 angle of repose analysis.

| | Height of | Base of | Angle of |
|---|---|---|---|
| Test | Cone (cm) | Cone (cm) | Repose |
| 1 | 4 | 11.1 | 35.8 |
| 2 | 3.2 | 12 | 28.1 |
| 3 | 3.3 | 11.8 | 29.2 |

| | |
|---|---|
| Average | 31.0 |
| % RSD | 13.4 |

Formulation 21003-1 was not tested using a FLOWDEX.

iii. 21003-2

The nut flour composition of formulation 21003-2 in a 2000 g batch size is shown in Table G.

TABLE G
21003-2 nut flour composition.

| Active Pharmaceutical | Protein content | | Purity |
|---|---|---|---|
| Ingredient (API) | (HPLC) | Water | factor |
| Cashew flour | 32 | 4.1 | 0.30688 |
| Hazelnut flour | 38 | 4.9 | 0.36138 |
| Walnut flour | 47 | 4.4 | 0.44932 |
| Peanut flour | 50 | 2.4 | 0.488 |

Formulation 21003-2 was prepared with STARCAP 1500, AVICEL PH-101, CAB-O-SIL M-5P, and magnesium stearate. The descriptions of each of these excipients are provided in formulation 21003-1, above.

To generate the 21003-2 formulation, 1267.76 g of APIs and 10.00 g of CAB-O-SIL M-5P were bag blended. The resulting blend was milled through a 22R CNC Router and a PHILIPS TWIST HR-1701. A ¼ geometric blend with a 500 g batch size was then produced by mixing 168.50 g AVICEL PH-101 (168.50 g blender charge), 50 g STARCAP 1500 (218.50 g blender charge), 113.01 g of the blended API+CAB-O-SIL M-5P mixture (331.50 g blender charge), and another 168.50 g AVICEL PH-101 (500.00 g blender charge), in that order. The resulting ¼ geometric blend was milled through a 32R CNC Router and a PHILIPS TWIST HR-1701. The other excipients were then adjusted to the yield. A main blend was then produced by mixing 670.00 g AVICEL PH-101 (670.00 g blender charge), 150.00 g STARCAP 1500 (820.00 g blender charge), 500.00 g of the ¼ geometric blend (1320.00 g blender charge), and another 670.00 g AVICEL PH-101 (1990.00 g blender charge), in that order. The resulting main blend was milled through a 32R CNC Router and a PHILIPS TWIST HR-1701. The main blend was lubricated, thus resulting in the final main blend #2 21003-1 formulation.

A summary of the composition of a 1200 mg dosage of formulation 21003-2 is shown in Table H.

TABLE H
Formulation 21003-2 composition.

| | 1200 mg Dosage 21003-2 | | | |
|---|---|---|---|---|
| | | | Adjusted | Adjusted |
| Ingredient | °/Ow/w | mg/dose | g/batch | °/Ow/w |
| Cashew flour (Lot: | 6.15 | 300.00 | 401.06 | 20.05 |
| 21002-17) | | | | |
| Hazelnut flour (Lot: | 6.15 | 300.00 | 340.57 | 17.03 |
| 21002-20) | | | | |
| Walnut flour (Lot: | 6.15 | 300.00 | 273.92 | 13.70 |
| 21002-23) | | | | |
| Peanut Flour (Lot: | 6.15 | 300.00 | 252.21 | 12.61 |
| 121FA04020) | | | | |
| STARCAP 1500 | 10.00 | 487.50 | 200.00 | 10.00 |
| AVICEL PH-101 | 64.38 | 3138.75 | 512.24 | 25.61 |
| CAB-O-SIL M-5P | 0.50 | 24.38 | 10.00 | 0.50 |
| Magnesium stearate | 0.50 | 24.38 | 10.00 | 0.50 |
| | 100.00 | 4875.00 | 2000.00 | 100.00 |

Table I below shows the results of the density analysis of formulation 21003-2.

TABLE I
Formulation 21003-2 density analysis.

| | | | |
|---|---|---|---|
| Material weight (g) | 68.9 | Material weight (g) | 68.90 |
| Number of taps | 2500 | Bulk density (g/mL) | 0.30 |
| Bulk volume (mL) | 232 | Tapped density (g/mL) | 0.59 |
| Tapped Volume (mL) | 116 | Carr's index | 50.00 |

The density analysis showed that formulation 21003-2 has a Carr's index of 37.40. The angle of repose analysis of formulation 21003-2 is shown in Table J.

TABLE J
Formulation 21003-2 angle of repose analysis.

| | Height of | Base of | Angle of |
|---|---|---|---|
| Test | Cone (cm) | Cone (cm) | Repose |
| 1 | 6.2 | 12.9 | 43.9 |
| 2 | 6.3 | 13.2 | 43.7 |
| 3 | 6.4 | 12.8 | 45.0 |

| | |
|---|---|
| Average | 44.2 |
| % RSD | 1.6 |

The FLOWDEX results indicated that formulation 21003-2 did not pass through a 34 mm opening.

iv. 21003-3

The nut flour composition of formulation 21003-3 in a 50 g batch size is shown in Table K.

TABLE K
21003-3 nut flour composition.

| Active Pharmaceutical | Protein content | | |
|---|---|---|---|
| Ingredient (API) | (HPLC) | Water | Purity factor |
| Cashew flour | 32 | 4.1 | 0.30688 |
| Hazelnut flour | 38 | 4.9 | 0.36138 |
| Walnut flour | 47 | 4.4 | 0.44932 |
| Peanut flour | 50 | 2.4 | 0.488 |

Formulation 21003-3 was prepared with STARCAP, PARTEK SI 150, PARTEK M200, CAB-O-SIL M-5P, and magnesium stearate. CAB-O-SIL M-5P and magnesium stearate are described in formulation 21003-1, above.

STARCAP is a partially pregelatinized starch with an optimized particle morphology to facilitate excellent flow in your capsule dosage for. It has a bulk density of 0.42, a tapped density of 0.58, a median particle diameter (D50) of ~90 μm.

PARTEK SI 150 (sorbitol) has a D50 of 1.49 g/cm³, a bulk density of 450 kg/cm³, and a solubility of 2350 g/L. It is a directly compressible sorbitol with an excellent tableting behavior and a pleasant mouthfeel and taste. Moreover, it enables fast disintegration and dissolution, even at high tablet hardnesses, and it is well-suitable for low dose API formulations as it supports a good content uniformity.

PARTEK M200 (mannitol) is a spray dried mannitol, with a D50 of 1.52 g/cm³, a bulk density of 400-500 kg/cm³, and a solubility of 213 g/L. It is specifically for solid oral dose applications, and is a directly compressible mannitol with excellent compressibility which keeps the API stable and enables rapid disintegration and fast dissolution.

A summary of the composition of a 1200 mg dosage of formulation 21003-3 is shown in Table L.

TABLE L
Formulation 21003-3 composition.

| | 1200 mg Dosage 21003-3 | | | | |
|---|---|---|---|---|---|
| | | | Adjusted | Adjusted | Adjusted |
| Ingredient | % w/w | mg/dose | g/batch | % w/w | mg/dose |
| Cashew flour | 6.15 | 300.00 | 10.03 | 20.05 | 977.58 |
| (Lot: 21002-17) | | | | | |
| Hazelnut flour | 6.15 | 300.00 | 8.51 | 17.03 | 830.15 |
| (Lot: 21002-20) | | | | | |
| Walnut flour | 6.15 | 300.00 | 6.85 | 13.70 | 667.68 |
| (Lot: 21002-23) | | | | | |
| Peanut Flour | 6.15 | 300.00 | 6.31 | 12.61 | 614.75 |
| (Lot: 121FA04020) | | | | | |
| STARCAP | 10.00 | 487.50 | 5.00 | 10.00 | 487.50 |
| PARTEK SI 150 | 10.00 | 487.50 | 5.00 | 10.00 | 487.50 |
| PARTEK M200 | 52.88 | 2578.13 | 7.06 | 14.11 | 687.96 |
| CAB-O-SIL M-5P | 2.00 | 97.50 | 1.00 | 2.00 | 97.50 |
| Magnesium stearate | 0.50 | 24.38 | 0.25 | 0.50 | 24.38 |
| | 100.00 | 4875.00 | 50.00 | 100.00 | 4875.00 |

Table M below shows the results of the density analysis of formulation 21003-3.

TABLE M
Formulation 21003-3 density analysis.

| | | | |
|---|---|---|---|
| Material weight (g) | 34.895 | Material weight (g) | 34.90 |
| Number of taps | 2500 | Bulk density (g/mL) | 0.35 |
| Bulk volume (mL) | 100 | Tapped density (g/mL) | 0.62 |
| Tapped Volume (mL) | 56 | Carr's index | 44.00 |

The density analysis showed that formulation 21003-3 has a Carr's index of 44.00. The angle of repose analysis of formulation 21003-3 is shown in Table N.

TABLE N
Formulation 21003-3 angle of repose analysis.

| Test | Height of Cone (cm) | Base of Cone (cm) | Angle of Repose |
|---|---|---|---|
| 1 | 5.6 | 12.7 | 41.4 |
| 2 | 5.7 | 12.3 | 42.8 |
| 3 | 5.9 | 12.4 | 43.6 |

| | |
|---|---|
| Average | 42.6 |
| % RSD | 2.6 |

The FLOWDEX results indicated that formulation 21003-3 passes through a 34 mm opening.

v. 21003-4

The nut flour composition of formulation 21003-4 in a 50 g batch size is shown in Table O.

TABLE O
21003-4 nut flour composition.

| Active Pharmaceutical | Protein content | | |
|---|---|---|---|
| Ingredient (API) | (HPLC) | Water | Purity factor |
| Cashew flour | 32 | 4.1 | 0.30688 |
| Hazelnut flour | 38 | 4.9 | 0.36138 |
| Walnut flour | 47 | 4.4 | 0.44932 |
| Peanut flour | 50 | 2.4 | 0.488 |

Formulation 21003-4 was prepared with STARCAP, CAB-O-SIL M-5P, and magnesium stearate. STARCAP is described in formulation 21003-3, above, and CAB-O-SIL M-5P and magnesium stearate are described in formulation 21003-1, above.

A summary of the composition of a 1200 mg dosage of formulation 21003-4 is shown in Table P.

TABLE P
Formulation 21003-4 composition.

| | 1200 mg Dosage 21003-4 | | | | |
|---|---|---|---|---|---|
| | | | Adjusted | Adjusted | Adjusted |
| Ingredient | % w/w | mg/dose | g/batch | % w/w | mg/dose |
| Cashew flour | 6.15 | 300.00 | 10.03 | 20.05 | 977.58 |
| (Lot: 21002-17) | | | | | |
| Hazelnut flour | 6.15 | 300.00 | 8.51 | 17.03 | 830.15 |
| (Lot: 21002-20) | | | | | |
| Walnut flour | 6.15 | 300.00 | 6.85 | 13.70 | 667.68 |
| (Lot: 21002-23) | | | | | |
| Peanut Flour | 6.15 | 300.00 | 6.31 | 12.61 | 614.75 |
| (Lot: 121FA04020) | | | | | |
| STARCAP | 72.88 | 3553.13 | 17.06 | 34.11 | 1662.96 |
| CAB-O-SIL M-5P | 2.00 | 97.50 | 1.00 | 2.00 | 97.50 |
| Magnesium stearate | 0.50 | 24.38 | 0.25 | 0.50 | 24.38 |
| | 100.00 | 4875.00 | 50.00 | 100.00 | 4875.00 |

Table Q below shows the results of the density analysis of formulation 21003-4.

TABLE Q
Formulation 21003-4 density analysis.

| | | | |
|---|---|---|---|
| Material weight (g) | 31.377 | Material weight (g) | 31.38 |
| Number of taps | 2500 | Bulk density (g/mL) | 0.32 |
| Bulk volume (mL) | 98 | Tapped density (g/mL) | 0.60 |
| Tapped Volume (mL) | 52 | Carr's index | 46.94 |

The density analysis showed that formulation 21003-4 has a Carr's index of 46.94. The angle of repose analysis of formulation 21003-4 is shown in Table R.

TABLE R
Formulation 21003-4 angle of repose analysis.

| Test | Height of Cone (cm) | Base of Cone (cm) | Angle of Repose |
|---|---|---|---|
| 1 | 6.2 | 12.4 | 45.0 |
| 2 | 5.7 | 11.8 | 44.0 |
| 3 | 6.6 | 12.6 | 46.3 |

| | |
|---|---|
| Average | 45.1 |
| % RSD | 2.6 |

The FLOWDEX results indicated that formulation 21003-4 does not pass through a 34 mm opening.

vi. 21003-5

The nut flour composition of formulation 21003-5 in a 50 g batch size is shown in Table S.

TABLE S
21003-5 nut flour composition.

| Active Pharmaceutical | Protein content | | |
|---|---|---|---|
| Ingredient (API) | (HPLC) | Water | Purity factor |
| Cashew flour | 32 | 4.1 | 0.30688 |
| Hazelnut flour | 38 | 4.9 | 0.36138 |
| Walnut flour | 47 | 4.4 | 0.44932 |
| Peanut flour | 50 | 2.4 | 0.488 |

Formulation 21003-5 was prepared with GALENIQ 720 (isomalt), CAB-O-SIL M-5P, and magnesium stearate. CAB-O-SIL M-5P and magnesium stearate are described in formulation 21003-1, above.

GALENIQ 720 (isomalt) is a spray dried sorbitol/mannitol co-processed excipient produced from sucrose, with a bulk density of 0.4, a tapped density of 0.448, a D50 of ~180 μm. GALENIQ 720 has a pleasant sugar-like, natural taste profile with a sweetening profile almost equal to sucrose. It has very low hygroscopic, is chemically extremely stable, and has excellent flow properties.

A summary of the composition of a 1200 mg dosage of formulation 21003-5 is shown in Table T.

TABLE T
Formulation 21003-5 composition.

| | 1200 mg Dosage 21003-5 | | | | |
|---|---|---|---|---|---|
| | | | Adjusted | Adjusted | Adjusted |
| Ingredient | % w/w | mg/dose | g/batch | % w/w | mg/dose |
| Cashew flour | 6.15 | 300.00 | 10.03 | 20.05 | 977.58 |
| (Lot: 21002-17) | | | | | |
| Hazelnut flour | 6.15 | 300.00 | 8.51 | 17.03 | 830.15 |
| (Lot: 21002-20) | | | | | |
| Walnut flour | 6.15 | 300.00 | 6.85 | 13.70 | 667.68 |
| (Lot: 21002-23) | | | | | |
| Peanut Flour | 6.15 | 300.00 | 6.31 | 12.61 | 614.75 |
| (Lot: 121FA04020) | | | | | |
| GALENIQ 720 | 72.88 | 3553.13 | 17.06 | 34.11 | 1662.96 |
| (isomalt) | | | | | |
| CAB-O-SIL M-5P | 2.00 | 97.50 | 1.00 | 2.00 | 97.50 |
| Magnesium stearate | 0.50 | 24.38 | 0.25 | 0.50 | 24.38 |
| | 100.00 | 4875.00 | 50.00 | 100.00 | 4875.00 |

Table U below shows the results of the density analysis of formulation 21003-5.

TABLE U
Formulation 21003-5 density analysis.

| | | | |
|---|---|---|---|
| Material weight (g) | 29.094 | Material weight (g) | 29.09 |
| Number of taps | 2500 | Bulk density (g/mL) | 0.31 |
| Bulk volume (mL) | 93 | Tapped density (g/mL) | 0.58 |
| Tapped Volume (mL) | 50 | Carr's index | 46.24 |

The density analysis showed that formulation 21003-5 has a Carr's index of 46.24. The angle of repose analysis of formulation 21003-5 is shown in Table V.

TABLE V
Formulation 21003-5 angle of repose analysis.

| Test | Height of Cone (cm) | Base of Cone (cm) | Angle of Repose |
|---|---|---|---|
| 1 | 6.4 | 12.2 | 46.4 |
| 2 | 6.2 | 12.2 | 45.5 |
| 3 | 6.6 | 12.3 | 47.0 |

| | |
|---|---|
| Average | 46.3 |
| % RSD | 1.7 |

The FLOWDEX results indicated that formulation 21003-5 does not pass through a 34 mm opening.

vii. 21003-6

The nut flour composition of formulation 21003-6 in a 50 g batch size is shown in Table W.

TABLE W
21003-6 nut flour composition.

| Active Pharmaceutical | Protein content | | |
|---|---|---|---|
| Ingredient (API) | (HPLC) | Water | Purity factor |
| Cashew flour | 32 | 4.1 | 0.30688 |
| Hazelnut flour | 38 | 4.9 | 0.36138 |
| Walnut flour | 47 | 4.4 | 0.44932 |
| Peanut flour | 50 | 2.4 | 0.488 |

Formulation 21003-6 was prepared with PARTEK M200, CAB-O-SIL M-5P, and magnesium stearate. The description of PARTEK M200 is provided in formulation 21003-3, above, and the descriptions of CAB-O-SIL M-5 and magnesium stearate are provided in formulation 21003-1, above.

A summary of the composition of a 1200 mg dosage of formulation 21003-6 is shown in Table X.

TABLE X
Formulation 21003-6 composition.

| | 1200 mg Dosage 21003-6 | | | | |
|---|---|---|---|---|---|
| | | | Adjusted | Adjusted | Adjusted |
| Ingredient | % w/w | mg/dose | g/batch | % w/w | mg/dose |
| Cashew flour | 6.15 | 300.00 | 10.03 | 20.05 | 977.58 |
| (Lot: 21002-17) | | | | | |
| Hazelnut flour | 6.15 | 300.00 | 8.51 | 17.03 | 830.15 |
| (Lot: 21002-20) | | | | | |
| Walnut flour | 6.15 | 300.00 | 6.85 | 13.70 | 667.68 |
| (Lot: 21002-23) | | | | | |
| Peanut Flour | 6.15 | 300.00 | 6.31 | 12.61 | 614.75 |
| (Lot: 121FA04020) | | | | | |
| PARTEK M200 | 72.88 | 3553.13 | 17.06 | 34.11 | 1662.96 |
| CAB-O-SIL M-5P | 2.00 | 97.50 | 1.00 | 2.00 | 97.50 |
| Magnesium stearate | 0.50 | 24.38 | 0.25 | 0.50 | 24.38 |
| | 100.00 | 4875.00 | 50.00 | 100.00 | 4875.00 |

Table Y below shows the results of the density analysis of formulation 21003-6.

TABLE Y
Formulation 21003-6 density analysis.

| | | | |
|---|---|---|---|
| Material weight (g) | 29.424 | Material weight (g) | 29.42 |
| Number of taps | 2500 | Bulk density (g/mL) | 0.31 |
| Bulk volume (mL) | 95 | Tapped density (g/mL) | 0.58 |
| Tapped Volume (mL) | 51 | Carr's index | 46.32 |

The density analysis showed that formulation 21003-6 has a Carr's index of 46.32. The angle of repose analysis of formulation 21003-6 is shown in Table Z.

TABLE Z
Formulation 21003-6 angle of repose analysis.

| Test | Height of Cone (cm) | Base of Cone (cm) | Angle of Repose |
|---|---|---|---|
| 1 | 6.2 | 11.8 | 46.4 |
| 2 | 6.2 | 11.8 | 46.4 |
| 3 | 6.2 | 11.8 | 46.4 |

| | |
|---|---|
| Average | 46.4 |
| % RSD | 0.0 |

The FLOWDEX results indicated that formulation 21003-6 does not pass through a 34 mm opening.

viii. 21003-7

The nut flour composition of formulation 21003-7 in a 50 g batch size is shown in Table AA.

TABLE AA
21003-7 nut flour composition.

| Active Pharmaceutical | Protein content | | |
|---|---|---|---|
| Ingredient (API) | (HPLC) | Water | Purity factor |
| Cashew flour | 32 | 4.1 | 0.30688 |
| Hazelnut flour | 38 | 4.9 | 0.36138 |
| Walnut flour | 47 | 4.4 | 0.44932 |
| Peanut flour | 50 | 2.4 | 0.488 |

Formulation 21003-7 was prepared with DI-PAC granulated sugar, CAB-O-SIL M-5P, and magnesium stearate. The descriptions of CAB-O-SIL M-5P and magnesium stearate are provided in formulation 21003-1, above.

DI-PAC granulated sugar is a direct compacting and tabling sugar, containing sucrose (97%) and maltodextrin (3%). It is highly compressible, readily soluble, does not contain fructose nor invert sugar, is inert, has low hygroscopicity and low moisture content, is a good dispersing agent and porous to allow for uniform distribution of active ingredients.

A summary of the composition of a 1200 mg dosage of formulation 21003-7 is shown in Table BB.

TABLE BB
Formulation 21003-7 composition.

| | 1200 mg Dosage 21003-7 | | | | |
|---|---|---|---|---|---|
| | | | Adjusted | Adjusted | Adjusted |
| Ingredient | % w/w | mg/dose | g/batch | % w/w | mg/dose |
| Cashew flour (Lot: 21002-17) | 6.15 | 300.00 | 10.03 | 20.05 | 977.58 |
| Hazelnut flour (Lot: 21002-20) | 6.15 | 300.00 | 8.51 | 17.03 | 830.15 |
| Walnut flour (Lot: 21002-23) | 6.15 | 300.00 | 6.85 | 13.70 | 667.68 |
| Peanut Flour (Lot: 121FA04020) | 6.15 | 300.00 | 6.31 | 12.61 | 614.75 |
| DI-PAC | 72.88 | 3553.13 | 17.06 | 34.11 | 1662.96 |
| CAB-O-SIL M-5P | 2.00 | 97.50 | 1.00 | 2.00 | 97.50 |
| Magnesium stearate | 0.50 | 24.38 | 0.25 | 0.50 | 24.38 |
| | 100.00 | 4875.00 | 50.00 | 100.00 | 4875.00 |

Table CC below shows the results of the density analysis of formulation 21003-7.

TABLE CC
Formulation 21003-7 density analysis.

| | | | |
|---|---|---|---|
| Material weight (g) | 31.79 | Material weight (g) | 31.79 |
| Number of taps | 2500 | Bulk density (g/mL) | 0.34 |
| Bulk volume (mL) | 94 | Tapped density (g/mL) | 0.64 |
| Tapped Volume (mL) | 50 | Carr's index | 46.81 |

The density analysis showed that formulation 21003-7 has a Carr's index of 46.81. The angle of repose analysis of formulation 21003-7 is shown in Table DD.

TABLE DD
Formulation 21003-7 angle of repose analysis.

| Test | Height of Cone (cm) | Base of Cone (cm) | Angle of Repose |
|---|---|---|---|
| 1 | 6 | 11.9 | 45.2 |
| 2 | 6 | 12 | 45.0 |
| 3 | 6.2 | 12.4 | 45.0 |

| | |
|---|---|
| Average | 45.1 |
| % RSD | 0.3 |

The FLOWDEX results indicated that formulation 21003-7 does not pass through a 34 mm opening.

ix. 21003-8

The nut flour composition of formulation 21003-8 in a 50 g batch size is shown in Table EE.

TABLE EE
21003-8 nut flour composition.

| Active Pharmaceutical | Protein content | | |
|---|---|---|---|
| Ingredient (API) | (HPLC) | Water | Purity factor |
| Cashew flour | 32 | 4.1 | 0.30688 |
| Hazelnut flour | 38 | 4.9 | 0.36138 |
| Walnut flour | 47 | 4.4 | 0.44932 |
| Peanut Flour | 50 | 2.4 | 0.488 |

Formulation 21003-8 was prepared with STARCAP, PARTEK SI 150, PARTEK M200, CAB-O-SIL M-5P, and magnesium stearate. STARCAP, PARTEK SI 150, and PARTEK M200 are described in formulation 21003-3, above, and CAB-O-SIL M-5P and magnesium stearate are described in formulation 21003-1, above.

Formulation 21003-8 is the same as formulation 21003-3, but it has been diluted to a 80 mg dosage. A summary of the composition of a 80 mg dosage of formulation 21003-8 is shown in Table FF.

TABLE FF
Formulation 21003-8 composition.

| | 80 mg Dosage 21003-8 | | | | |
|---|---|---|---|---|---|
| | | | Adjusted | Adjusted | Adjusted |
| Ingredient | % w/w | mg/dose | g/batch | % w/w | mg/dose |
| Cashew flour (Lot: 21002-17) | 3.24 | 20.00 | 5.27 | 10.55 | 65.17 |
| Hazelnut flour (Lot: 21002-20) | 3.24 | 20.00 | 4.48 | 8.96 | 55.34 |
| Walnut flour (Lot: 21002-23) | 3.24 | 20.00 | 3.60 | 7.20 | 44.51 |
| Peanut Flour (Lot: 121FA04020) | 3.24 | 20.00 | 3.32 | 6.63 | 40.98 |
| STARCAP | 20.00 | 123.60 | 10.00 | 20.00 | 123.60 |
| PARTEK SI 150 | 20.00 | 123.60 | 10.00 | 20.00 | 123.60 |
| PARTEK M200 | 44.56 | 275.35 | 12.08 | 24.16 | 149.34 |
| CAB-O-SIL M-5P | 2.00 | 12.36 | 1.00 | 2.00 | 12.36 |
| Magnesium stearate | 0.50 | 3.09 | 0.25 | 0.50 | 3.09 |
| | 100.00 | 618.00 | 50.00 | 100.00 | 618.00 |

25 g of formulation 21003-3 was diluted with 7.5 g STARCAP, 7.5 g PARTEK SI 150, 8.55 g PARTEK M200, 0.5 g CAB-O-SIL M-5P, and 0.13 g magnesium stearate to produce 21003-8.

Table GG below shows the results of the density analysis of formulation 21003-8.

TABLE GG
Formulation 21003-8 density analysis.

| | | | |
|---|---|---|---|
| Material weight (g) | 43.727 | Material weight (g) | 43.73 |
| Number of taps | 2500 | Bulk density (g/mL) | 0.47 |
| Bulk volume (mL) | 94 | Tapped density (g/mL) | 0.75 |
| Tapped Volume (mL) | 58 | Carr's index | 38.30 |

The density analysis showed that formulation 21003-8 has a Carr's index of 38.30. The angle of repose analysis of formulation 21003-8 is shown in Table HH.

TABLE HH
Formulation 21003-8 angle of repose analysis.

| Test | Height of Cone (cm) | Base of Cone (cm) | Angle of Repose |
|---|---|---|---|
| 1 | 3.9 | 10 | 38.0 |
| 2 | 3.9 | 10.5 | 36.6 |
| 3 | 4.1 | 9.9 | 39.6 |

| | |
|---|---|
| Average | 38.1 |
| % RSD | 4.0 |

The FLOWDEX results indicated that formulation 21003-8 passes through a 24 mm opening.

x. 21003-9

The nut flour composition of formulation 21003-9 in a 50 g batch size is shown in Table II.

TABLE II
21003-9 nut flour composition.

| Active Pharmaceutical | Protein content | | |
|---|---|---|---|
| Ingredient (API) | (HPLC) | Water | Purity factor |
| Cashew flour | 32 | 4.1 | 0.30688 |
| Hazelnut flour | 38 | 4.9 | 0.36138 |
| Walnut flour | 47 | 4.4 | 0.44932 |
| Peanut flour | 50 | 2.4 | 0.488 |

Formulation 21003-9 was prepared with STARCAP, PARTEK SI 150, PARTEK M200, Micronized Talc, CAB-O-SIL M-5P, and magnesium stearate. STARCAP, PARTEK SI 150, and PARTEK M200 are described in formulation 21003-3, above, and CAB-O-SIL M-5P and magnesium stearate are described in formulation 21003-1, above.

Micronized Talc is a white, moisture less chemical that is processed from magnesium-silicate-hydrate.

A summary of the composition of a 1200 mg dosage of formulation 21003-9 is shown in Table JJ.

TABLE JJ
Formulation 21003-9 composition.
Ingredient 1200 mg Dosage 21003-9

| | 1200 mg Dosage 21003-9 | | | | |
|---|---|---|---|---|---|
| | | | Adjusted | Adjusted | Adjusted |
| Ingredient | % w/w | mg/dose | g/batch | % w/w | mg/dose |
| Cashew flour (Lot: 21002-17) | 6.15 | 300.00 | 10.03 | 20.05 | 977.58 |
| Hazelnut flour (Lot: 21002-20) | 6.15 | 300.00 | 8.51 | 17.03 | 830.15 |
| Walnut flour (Lot: 21002-23) | 6.15 | 300.00 | 6.85 | 13.70 | 667.68 |
| Peanut Flour (Lot: 121FA04020) | 6.15 | 300.00 | 6.31 | 12.61 | 614.75 |
| STARCAP | 9.00 | 438.75 | 4.50 | 9.00 | 438.75 |
| PARTEK SI 150 | 9.00 | 438.75 | 4.50 | 9.00 | 438.75 |
| PARTEK M200 | 51.38 | 2505.00 | 6.31 | 12.61 | 614.84 |
| Micronized Talc | 5.00 | 243.75 | 2.50 | 5.00 | 243.75 |
| CAB-O-SIL M-5P | 0.50 | 24.38 | 0.25 | 0.50 | 24.38 |
| Magnesium stearate | 0.50 | 24.38 | 0.25 | 0.50 | 24.38 |
| | 100.00 | 4875.0 | 50.00 | 100.00 | 4875.00 |

Table KK below shows the results of the density analysis of formulation 21003-9.

TABLE KK
Formulation 21003-9 density analysis.

| | | | |
|---|---|---|---|
| Material weight (g) | 31.776 | Material weight (g) | 31.78 |
| Number of taps | 2500 | Bulk density (g/mL) | 0.32 |
| Bulk volume (mL) | 100 | Tapped density (g/mL) | 0.62 |
| Tapped Volume (mL) | 51 | Carr's index | 49.00 |

The density analysis showed that formulation 21003-9 has a Carr's index of 49.00. The angle of repose analysis of formulation 21003-9 is shown in Table LL.

TABLE LL
Formulation 21003-9 angle of repose analysis.

| Test | Height of Cone (cm) | Base of Cone (cm) | Angle of Repose |
|---|---|---|---|
| 1 | 5.8 | 12.2 | 43.6 |
| 2 | 5.8 | 12.3 | 43.3 |
| 3 | 5.9 | 12.1 | 44.3 |

| | |
|---|---|
| Average | 43.7 |
| % RSD | 1.1 |

The FLOWDEX results indicated that formulation 21003-9 does not pass through a 34 mm opening.

xi. 21003-10

The nut flour composition of formulation 21003-10 in a 50 g batch size is shown in Table MM.

TABLE MM
21003-10 nut flour composition.

| Active Pharmaceutical | Protein content | | |
|---|---|---|---|
| Ingredient (API) | (HPLC) | Water | Purity factor |
| Cashew flour | 32 | 4.1 | 0.30688 |
| Hazelnut flour | 38 | 4.9 | 0.36138 |
| Walnut flour | 47 | 4.4 | 0.44932 |
| Peanut flour | 50 | 2.4 | 0.488 |

Formulation 21003-10 was prepared with STARCAP, PARTEK SI 150, PEARLITOL SD200, CAB-O-SIL M-5P, and magnesium stearate. STARCAP and PARTEK SI 150 are described in formulation 21003-3, above, and CAB-O-SIL M-5P and magnesium stearate are described in formulation 21003-1, above.

PEARLITOL SD200 (mannitol) is used as a filler and filler/binder as well as a bulk sweetener, with a bulk density of 0.48, a tapped density of 0.58, and a D50 of ~170 μm. The mouthfeel is slightly sweet (half as sweet as sucrose), has a slight cooling effect in the mouth, and may be used to improve the overall mouthfeel of formulations in which it is used. PEARLITOL SD200 is stable in solid and solution states, and is not hygroscopic

A summary of the composition of a 1200 mg dosage of formulation 21003-10 is shown in Table NN.

TABLE NN
Formulation 21003-10 composition.

| | 1200 mg Dosage 21003-10 | | | | |
|---|---|---|---|---|---|
| | | | Adjusted | Adjusted | Adjusted |
| Ingredient | % w/w | mg/dose | g/batch | % w/w | mg/dose |
| Cashew flour (Lot: 21002-17) | 6.15 | 300.00 | 10.03 | 20.05 | 977.58 |
| Hazelnut flour (Lot: 21002-20) | 6.15 | 300.00 | 8.51 | 17.03 | 830.15 |
| Walnut flour (Lot: 21002-23) | 6.15 | 300.00 | 6.85 | 13.70 | 667.68 |
| Peanut Flour (Lot: 121FA04020) | 6.15 | 300.00 | 6.31 | 12.61 | 614.75 |
| STARCAP | 10.00 | 487.50 | 5.00 | 10.00 | 487.50 |
| PARTEK SI 150 | 10.00 | 487.50 | 5.00 | 10.00 | 487.50 |
| PEARLITOL SD200 | 52.88 | 2578.13 | 7.06 | 14.11 | 687.96 |
| CAB-O-SIL M-5P | 2.00 | 97.50 | 1.00 | 2.00 | 97.50 |
| Magnesium stearate | 0.50 | 24.38 | 0.25 | 0.50 | 24.38 |
| | 100.00 | 4875.00 | 50.00 | 100.00 | 4875.00 |

Table OO below shows the results of the density analysis of formulation 21003-10.

TABLE OO
Formulation 21003-10 density analysis.

| | | | |
|---|---|---|---|
| Material weight (g) | 33.142 | Material weight (g) | 33.14 |
| Number of taps | 2500 | Bulk density (g/mL) | 0.33 |
| Bulk volume (mL) | 99 | Tapped density | 0.60 |
| | | (g/mL) | |
| Tapped Volume | 55 | Carr's index | 44.44 |
| (mL) | | | |

The density analysis showed that formulation 21003-10 has a Carr's index of 44.44. The angle of repose analysis of formulation 21003-10 is shown in Table PP.

TABLE PP
Formulation 21003-10 angle of repose analysis.

| | Height of | Base of | Angle of |
|---|---|---|---|
| Test | Cone (cm) | Cone (cm) | Repose |
| 1 | 6 | 12.1 | 44.8 |
| 2 | 6.1 | 12.1 | 45.2 |
| 3 | 5.8 | 12 | 44.0 |

| | |
|---|---|
| Average | 44.7 |
| % RSD | 1.4 |

The FLOWDEX results indicated that formulation 21003-10 passes through a 34 mm opening.

xii. 21003-11

The nut flour composition of formulation 21003-11 in a 50 g batch size is shown in Table QQ.

TABLE QQ
21003-11 nut flour composition.

| Active Pharmaceutical | Protein content | | Purity |
|---|---|---|---|
| Ingredient (API) | (HPLC) | Water | factor |
| Cashew flour | 32 | 4.1 | 0.30688 |
| Hazelnut flour | 38 | 4.9 | 0.36138 |
| Walnut flour | 47 | 4.4 | 0.44932 |
| Peanut flour | 50 | 2.4 | 0.488 |

Formulation 21003-11 was prepared with STARCAP, PEARLITOL® SD200, CAB-O-SIL M-5P, and magnesium stearate. STARCAP is described in formulation 21003-3, above, PEARLITOL SD200 is described in formulation 21003-10, above, and CAB-O-SIL M-5P and magnesium stearate are described in formulation 21003-1, above.

A summary of the composition of a 1200 mg dosage of formulation 21003-11 is shown in Table RR.

TABLE RR
Formulation 21003-11 composition.

| | 1200 mg Dosage 21003-11 | | | | |
|---|---|---|---|---|---|
| | | | Adjusted | Adjusted % | Adjusted |
| Ingredient | % w/w | mg/dose | g/batch | w/w | mg/dose |
| Cashew flour (Lot: 21002-17) | 6.15 | 300.00 | 10.03 | 20.05 | 977.58 |
| Hazelnut flour (Lot: 21002-20) | 6.15 | 300.00 | 8.51 | 17.03 | 830.15 |
| Walnut flour (Lot: 21002-23) | 6.15 | 300.00 | 6.85 | 13.70 | 667.68 |
| Peanut Flour (Lot: 121FA04020) | 6.15 | 300.00 | 6.31 | 12.61 | 614.75 |
| STARCAP | 10.00 | 487.50 | 5.00 | 10.00 | 487.50 |
| PEARLITOL SD200 | 62.88 | 3065.63 | 12.06 | 24.11 | 1175.46 |
| CAB-O-SIL M-5P | 2.00 | 97.50 | 1.00 | 2.00 | 97.50 |
| Magnesium stearate | 0.50 | 24.38 | 0.25 | 0.50 | 24.38 |
| | 100.00 | 4875.00 | 50.00 | 100.00 | 4875.00 |

Table SS below shows the results of the density analysis of formulation 21003-11.

TABLE SS
Formulation 21003-11 density analysis.

| | | | |
|---|---|---|---|
| Material weight (g) | 32.068 | Material weight (g) | 32.07 |
| Number of taps | 2500 | Bulk density (g/mL) | 0.32 |
| Bulk volume (mL) | 99 | Tapped density (g/mL) | 0.57 |
| Tapped Volume (mL) | 56 | Carr's index | 43.43 |

The density analysis showed that formulation 21003-11 has a Carr's index of 43.43. The angle of repose analysis of formulation 21003-11 is shown in Table TT.

TABLE TT
Formulation 21003-11 angle of repose analysis.

| | Height of | Base of | Angle of |
|---|---|---|---|
| Test | Cone (cm) | Cone (cm) | Repose |
| 1 | 5.4 | 12.1 | 41.8 |
| 2 | 5.9 | 11.9 | 44.8 |
| 3 | 5.9 | 12 | 44.5 |

| | |
|---|---|
| Average | 43.7 |
| % RSD | 3.8 |

The FLOWDEX results indicated that formulation 21003-11 passes through a 34 mm opening.

xiii. 21003-12

The nut flour composition of formulation 21003-12 in a 50 g batch size is shown in Table UU.

TABLE UU
21003-12 nut flour composition.

| Active Pharmaceutical | Protein content | | Purity |
|---|---|---|---|
| Ingredient (API) | (HPLC) | Water | factor |
| Cashew flour | 32 | 4.1 | 0.30688 |
| Hazelnut flour | 38 | 4.9 | 0.36138 |
| Walnut flour | 47 | 4.4 | 0.44932 |
| Peanut flour | 50 | 2.4 | 0.488 |

Formulation 21003-12 was prepared with STARCAP, FASTFLO 316 (lactose), CAB-O-SIL® M-5P, and magnesium stearate. STARCAP is described in formulation 21003-3, above, and CAB-O-SIL M-5P and magnesium stearate are described in formulation 21003-1, above.

FAST FLO Lactose 316 is a spray-dried mixture of crystalline and amorphous lactose, which may be used for direct compression.

A summary of the composition of a 1200 mg dosage of formulation 21003-12 is shown in Table W.

TABLE VV
Formulation 21003-12 composition.

| | 1200 mg Dosage 21003-12 | | | | |
|---|---|---|---|---|---|
| | | | Adjusted | Adjusted % | Adjusted |
| Ingredient | % w/w | mg/dose | g/batch | w/w | mg/dose |
| Cashew flour (Lot: 21002-17) | 6.15 | 300.00 | 10.03 | 20.05 | 977.58 |
| Hazelnut flour (Lot: 21002-20) | 6.15 | 300.00 | 8.51 | 17.03 | 830.15 |
| Walnut flour (Lot: 21002-23) | 6.15 | 300.00 | 6.85 | 13.70 | 667.68 |
| Peanut Flour (Lot: 121FA04020) | 6.15 | 300.00 | 6.31 | 12.61 | 614.75 |
| STARCAP | 10.00 | 487.50 | 5.00 | 10.00 | 487.50 |
| FASTFLO 316 | 62.88 | 3065.63 | 12.06 | 24.11 | 1175.46 |
| CAB-O-SIL M-5P | 2.00 | 97.50 | 1.00 | 2.00 | 97.50 |
| Magnesium stearate | 0.50 | 24.38 | 0.25 | 0.50 | 24.38 |
| | 100.00 | 4875.00 | 50.00 | 100.00 | 4875.00 |

Table WW below shows the results of the density analysis of formulation 21003-12.

TABLE WW
Formulation 21003-12 density analysis.

| | | | |
|---|---|---|---|
| Material weight (g) | 33.011 | Material weight (g) | 33.01 |
| Number of taps | 2500 | Bulk density (g/mL) | 0.34 |
| Bulk volume (mL) | 98 | Tapped density (g/mL) | 0.60 |
| Tapped Volume (mL) | 55 | Carr's index | 43.88 |

The density analysis showed that formulation 21003-12 has a Carr's index of 43.88. The angle of repose analysis of formulation 21003-12 is shown in Table XX.

TABLE XX
Formulation 21003-12 angle of repose analysis.
Height of Cone (cm)

| | Height of | Base of | Angle of |
|---|---|---|---|
| Test | Cone (cm) | Cone (cm) | Repose |
| 1 | 5.9 | 12.8 | 42.7 |
| 2 | 6 | 12.3 | 44.3 |
| 3 | 6 | 12.3 | 44.3 |

| | |
|---|---|
| Average | 43.8 |
| % RSD | 2.1 |

The FLOWDEX results indicated that formulation 21003-12 passes through a 34 mm opening and a 32 mm opening, but does not pass through a 30 mm opening.

xiv. 21003-13

The nut flour composition of formulation 21003-13 in a 50 g batch size is shown in Table YY.

TABLE YY
21003-13 nut flour composition.

| Active Pharmaceutical | Protein content | | Purity |
|---|---|---|---|
| Ingredient (API) | (HPLC) | Water | factor |
| Cashew flour | 32 | 4.1 | 0.30688 |
| Hazelnut flour | 38 | 4.9 | 0.36138 |
| Walnut flour | 47 | 4.4 | 0.44932 |
| Peanut flour | 50 | 2.4 | 0.488 |

Formulation 21003-13 was prepared with STARCAP, GALENIQ 720 (isomalt), PEARLITOL SD200 (mannitol), AVICEL HFE-102, CAB-O-SIL M-5P, and magnesium stearate. STARCAP is described in formulation 21003-3, above, GALENIQ 720 (isomalt) is described in formulation 21003-4, above, and PEARLITOL SD200 is described above, and CAB-O-SIL M-5P and magnesium stearate are described in formulation 21003-1, above.

AVICEL HFE-102 is a spray dried blend comprised of 90% microcrystalline cellulose (MCC) and 10% mannitol, with a bulk density of 0.41, a tapped density of 0.51, and a D50 of ~100-130 μm. The mannitol/MCC combination improves palatability, decreases the disintegration time due to the good water solubility and wetting properties, and the mannitol confers a negative heat of solution.

A summary of the composition of a 1200 mg dosage of formulation 21003-13 is shown in Table ZZ.

TABLE ZZ
Formulation 21003-13 composition.

| | 1200 mg Dosage 21003-13 | | | | |
|---|---|---|---|---|---|
| | % | | Adjusted | Adjusted % | Adjusted |
| Ingredient | w/w | mg/dose | g/batch | w/w | mg/dose |
| Cashew flour (Lot: 21002-17) | 300.00 | 6.25 | 977.58 | 20.37 | 10.18 |
| Hazelnut flour (Lot: 21002-20) | 300.00 | 6.25 | 830.15 | 17.29 | 8.65 |
| Walnut flour (Lot: 21002-23) | 300.00 | 6.25 | 667.68 | 13.91 | 6.95 |
| Peanut Flour (Lot: 121FA04020) | 300.00 | 6.25 | 614.75 | 12.81 | 6.40 |
| STARCAP | 500.00 | 10.42 | 500.00 | 10.42 | 5.21 |
| GALENIQ 720 (isomalt) | 2430.00 | 50.63 | 539.84 | 11.25 | 5.62 |
| PEARLITOL SD200 | 150.00 | 3.13 | 150.00 | 3.13 | 1.56 |
| AVICEL HFE-102 | 400.00 | 8.33 | 400.00 | 8.33 | 4.17 |
| CAB-O-SIL M-5P | 96.00 | 2.00 | 96.00 | 2.00 | 1.00 |
| Magnesium stearate | 24.00 | 0.50 | 24.00 | 0.50 | 0.25 |
| | 4800.00 | 100.00 | 4800.00 | 100.00 | 50.00 |

Table AAA below shows the results of the density analysis of formulation 21003-13.

TABLE AAA
Formulation 21003-13 density analysis.

| | | | |
|---|---|---|---|
| Material weight (g) | 31.191 | Material weight (g) | 31.19 |
| Number of taps | 2500 | Bulk density (g/mL) | 0.33 |
| Bulk volume (mL) | 95 | Tapped density (g/mL) | 0.58 |
| Tapped Volume (mL) | 54 | Carr's index | 43.16 |

The density analysis showed that formulation 21003-13 has a Carr's index of 43.16. The angle of repose analysis of formulation 21003-13 is shown in Table BBB.

TABLE BBB
Formulation 21003-13 angle of repose analysis.

| | Height of | Base of | Angle of |
|---|---|---|---|
| Test | Cone (cm) | Cone (cm) | Repose |
| 1 | 6.1 | 12.9 | 43.4 |
| 2 | 6 | 12.8 | 43.2 |
| 3 | 6 | 12.8 | 43.2 |

| | |
|---|---|
| Average | 43.2 |
| % RSD | 0.3 |

The FLOWDEX results indicated that formulation 21003-13 passes through a 32 mm opening, but does not pass through a 30 mm opening.

xv. 21003-14

The nut flour composition of formulation 21003-14 in a 50 g batch size is shown in Table CCC.

TABLE CCC
21003-14 nut flour composition.

| Active Pharmaceutical | Protein content | | Purity |
|---|---|---|---|
| Ingredient (API) | (HPLC) | Water | factor |
| Cashew flour | 32 | 4.1 | 0.30688 |
| Hazelnut flour | 38 | 4.9 | 0.36138 |
| Walnut flour | 47 | 4.4 | 0.44932 |
| Peanut flour | 50 | 2.4 | 0.488 |

Formulation 21003-14 was prepared with STARCAP, GALENIQ 720 (isomalt), PEARLITOL SD200, AVICEL CE-15, CAB-O-SIL M-5P, and magnesium stearate. STARCAP is described in formulation 21003-3, above, GALENIQ 720 (isomalt) is described in formulation 21003-4, above, and PEARLITOL SD200 is described above, and CAB-O-SIL M-5P and magnesium stearate are described in formulation 21003-1, above.

AVICEL CE-15 is a spray dried blend comprising 85% MCC and 15% guar gum, with a bulk density of 0.5, a tapped density of 0.62, and a D50 of ~100 μm. It improves sensory attributes by reducing grittiness, tooth packing, and friability to create a creamier mouthfeel. AVICEL CE-15 is highly hygroscopic and weakly acidic.

A summary of the composition of a 1200 mg dosage of formulation 21003-14 is shown in Table DDD.

TABLE DDD
Formulation 21003-14 composition.

| | 1200 mg Dosage 21003-14 | | | | |
|---|---|---|---|---|---|
| | | | Adjusted | Adjusted | Adjusted |
| Ingredient | % w/w | mg/dose | g/batch | % w/w | mg/dose |
| Cashew flour (Lot: 21002-25) | 300.00 | 6.25 | 977.58 | 20.37 | 10.18 |
| Hazelnut flour (Lot: 21002-26) | 300.00 | 6.25 | 830.15 | 17.29 | 8.65 |
| Walnut flour (Lot: 21002-27) | 300.00 | 6.25 | 667.68 | 13.91 | 6.95 |
| Peanut Flour (Lot: 121FA04020) | 300.00 | 6.25 | 614.75 | 12.81 | 6.40 |
| STARCAP | 500.00 | 10.42 | 500.00 | 10.42 | 5.21 |
| GALENIQ 720 (isomalt) | 2430.00 | 50.63 | 539.84 | 11.25 | 5.62 |
| PEARLITOL SD200 | 150.00 | 3.13 | 150.00 | 3.13 | 1.56 |
| AVICEL CE-15 | 400.00 | 8.33 | 400.00 | 8.33 | 4.17 |
| CAB-O-SIL M-5P | 96.00 | 2.00 | 96.00 | 2.00 | 1.00 |
| Magnesium stearate | 24.00 | 0.50 | 24.00 | 0.50 | 0.25 |
| | 4800.00 | 100.00 | 4800.00 | 100.00 | 50.00 |

Table EEE below shows the results of the density analysis of formulation 21003-14.

TABLE EEE
Formulation 21003-14 density analysis.

| | | | |
|---|---|---|---|
| Material weight (g) | 55.807 | Material weight (g) | 55.81 |
| Number of taps | 2500 | Bulk density (g/mL) | 0.56 |
| Bulk volume (mL) | 100 | Tapped density (g/mL) | 1.24 |
| Tapped Volume (mL) | 45 | Carr's index | 55.00 |

The density analysis showed that formulation 21003-14 has a Carr's index of 55.00. The angle of repose analysis of formulation 21003-14 is shown in Table FFF.

TABLE FFF
Formulation 21003-14 angle of repose analysis.

| Test | Height of Cone (cm) | Base of Cone (cm) | Angle of Repose |
|---|---|---|---|
| 1 | 6.3 | 12.8 | 44.5 |
| 2 | 6.7 | 12.9 | 46.1 |
| 3 | 6.3 | 12.7 | 44.8 |

| | |
|---|---|
| Average | 45.1 |
| % RSD | 1.8 |

The FLOWDEX results indicated that formulation 21003-14 passes through a 32 mm opening, but does not pass through a 30 mm opening.

xvi. 21003-15

The nut flour composition of formulation 21003-15 in a 50 g batch size is shown in Table GGG.

TABLE GGG
21003-15 nut flour composition.

| Active Pharmaceutical | Protein content | | |
|---|---|---|---|
| Ingredient (API) | (HPLC) | Water | Purity factor |
| Cashew flour | 32 | 4.1 | 0.30688 |
| Hazelnut flour | 38 | 4.9 | 0.36138 |
| Walnut flour | 47 | 4.4 | 0.44932 |
| Peanut flour | 50 | 2.4 | 0.488 |

Formulation 21003-15 was prepared with STARCAP, GALENIQ 720 (isomalt), PEARLITOL SD200, CAB-O-SIL M-5P, and magnesium stearate. STARCAP is described in formulation 21003-3, above, GALENIQ 720 (isomalt) is described in formulation 21003-4, above, and PEARLITOL SD200 is described above, and CAB-O-SIL M-5P and magnesium stearate are described in formulation 21003-1, above.

A summary of the composition of a 1200 mg dosage of formulation 21003-15 is shown in Table HHH.

TABLE HHH
Formulation 21003-15 composition.

| | 1200 mg Dosage 21003-15 | | | | |
|---|---|---|---|---|---|
| | | | Adjusted | Adjusted | Adjusted |
| Ingredient | % w/w | mg/dose | g/batch | % w/w | mg/dose |
| Cashew flour (Lot: 21002-25) | 300.00 | 6.25 | 977.58 | 20.37 | 10.18 |
| Hazelnut flour (Lot: 21002-26) | 300.00 | 6.25 | 830.15 | 17.29 | 8.65 |
| Walnut flour (Lot: 21002-27) | 300.00 | 6.25 | 667.68 | 13.91 | 6.95 |
| Peanut Flour (Lot: 121FA04020) | 300.00 | 6.25 | 614.75 | 12.81 | 6.40 |
| STARCAP | 700.00 | 14.58 | 700.00 | 14.58 | 7.29 |
| GALENIQ 720 (isomalt) | 2580.00 | 53.75 | 689.84 | 14.37 | 7.19 |
| PEARLITOL SD200 | 200.00 | 4.17 | 200.00 | 4.17 | 2.08 |
| CAB-O-SIL M-5P | 96.00 | 2.00 | 96.00 | 2.00 | 1.00 |
| Magnesium stearate | 24.00 | 0.50 | 24.00 | 0.50 | 0.25 |
| | 4800.00 | 100.00 | 4800.00 | 100.00 | 50.00 |

Table III below shows the results of the density analysis of formulation 21003-15.

TABLE III
Formulation 21003-15 density analysis.

| | | | |
|---|---|---|---|
| Material weight (g) | 31.073 | Material weight (g) | 31.07 |
| Number of taps | 2500 | Bulk density (g/mL) | 0.32 |
| Bulk volume (mL) | 96 | Tapped density (g/mL) | 0.58 |
| Tapped Volume (mL) | 54 | Carr's index | 43.75 |

The density analysis showed that formulation 21003-15 has a Carr's index of 43.75. The angle of repose analysis of formulation 21003-15 is shown in Table JJJ.

TABLE JJJ
Formulation 21003-15 angle of repose analysis.

| Test | Height of Cone (cm) | Base of Cone (cm) | Angle of Repose |
|---|---|---|---|
| 1 | 6.2 | 13.1 | 43.4 |
| 2 | 6.2 | 13 | 43.6 |
| 3 | 6.3 | 12.9 | 44.3 |

| | |
|---|---|
| Average | 43.8 |
| % RSD | 1.1 |

The FLOWDEX results indicated that formulation 21003-15 passes through a 32 mm opening, but does not pass through a 30 mm opening.

xvii. 21003-17 (Milled cashew)

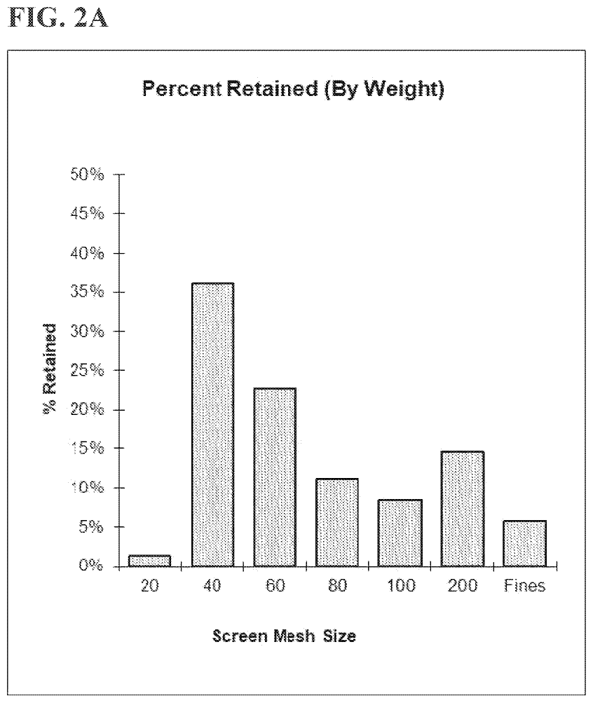

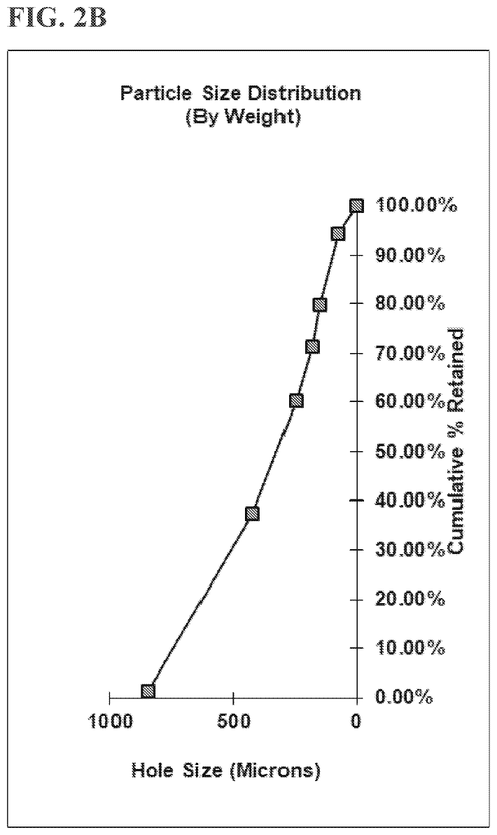

Formulation 21003-17 is a milled cashew formulation. Results of the sieve analysis of formulation 21003-17, with a total sample weight of 5.90 g, are shown in FIGS. 2A-2B, and Table KKK.

TABLE KKK
Formulation 21003-17 sieve analysis.

| | | Gross | Tare | Net | | |
|---|---|---|---|---|---|---|
| Mesh | Mesh size | weight | weight | weight | Percent | Cumulative |
| size | (microns) | (g) | (g) | (g) | retained | percent |
| 20 | 850 | 43.26 | 43.18 | 0.08 | 1.36% | 1.36% |
| 40 | 425 | 42.39 | 40.26 | 2.13 | 36.10% | 37.46% |
| 60 | 250 | 37.33 | 35.99 | 1.34 | 22.71% | 60.17% |
| 80 | 180 | 36.42 | 35.76 | 0.66 | 11.19% | 71.36% |
| 100 | 150 | 34.67 | 34.18 | 0.49 | 8.31% | 79.66% |
| 200 | 75 | 34.25 | 33.39 | 0.86 | 14.58% | 94.24% |
| Fine | 0 | 159.82 | 159.48 | 0.34 | 5.76% | 100.00% |

Table LLL below shows the results of the density analysis of Formulation 21003-17.

TABLE LLL
Formulation 21003-17 density analysis.

| | | | |
|---|---|---|---|
| Material weight (g) | 69.57 | Material weight (g) | 69.57 |
| Number of taps | 2500 | Bulk density (g/mL) | 0.29 |
| Bulk volume (mL) | 240 | Tapped density (g/mL) | 0.54 |
| Tapped Volume (mL) | 128 | Carr's index | 46.67 |

The density analysis showed that formulation 21003-17 has a Carr's index of 46.67. The angle of repose analysis of formulation 21003-17 is shown in Table MMM.

TABLE MMM
Formulation 21003-17 angle of repose analysis.

| Test | Height of Cone (cm) | Base of Cone (cm) | Angle of Repose |
|---|---|---|---|
| 1 | 5.8 | 12.4 | 43.1 |
| 2 | 5.6 | 12.7 | 41.4 |
| 3 | 5.7 | 12.9 | 41.5 |

| | |
|---|---|
| Average | 42.0 |
| % RSD | 2.3 |

xviii. 21003-20 (Milled hazelnut)

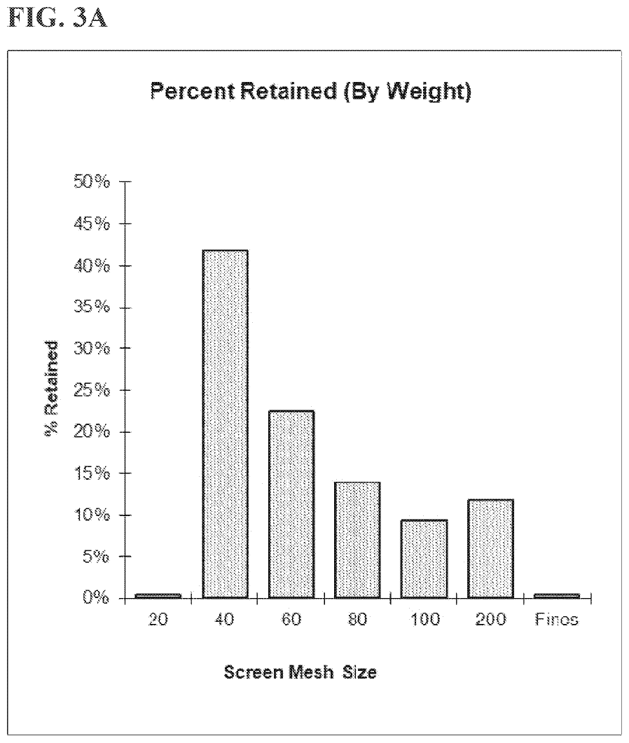

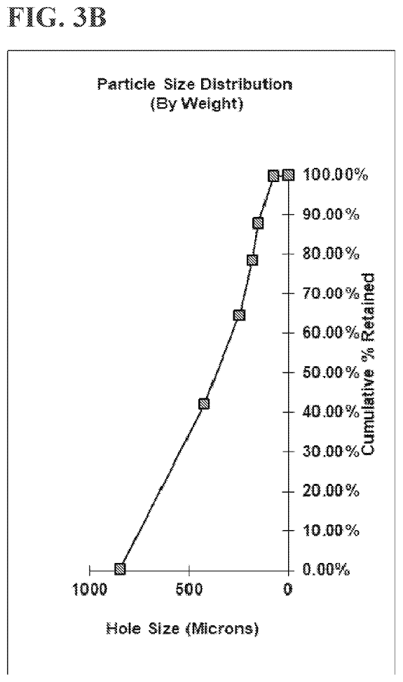

Formulation 21003-20 is a milled hazelnut formulation. Results of the sieve analysis of formulation 21003-20, with a total sample weight of 2.80 g, are shown in FIGS. 3A-3B, and Table NNN.

TABLE NNN
Formulation 21003-20 sieve analysis.

| | | Gross | Tare | Net | | |
|---|---|---|---|---|---|---|
| Mesh | Mesh size | weight | weight | weight | Percent | Cumulative |
| size | (microns) | (g) | (g) | (g) | retained | percent |
| 20 | 850 | 43.19 | 43.18 | 0.01 | 0.36% | 0.36% |
| 40 | 425 | 41.43 | 40.26 | 1.17 | 41.79% | 42.14% |
| 60 | 250 | 36.62 | 35.99 | 0.63 | 22.50% | 64.64% |
| 80 | 180 | 36.15 | 35.76 | 0.39 | 13.93% | 78.57% |
| 100 | 150 | 34.44 | 34.18 | 0.26 | 9.29% | 87.86% |
| 200 | 75 | 33.72 | 33.39 | 0.33 | 11.79% | 99.64% |
| Fines | 0 | 159.49 | 159.48 | 0.01 | 0.36% | 100.00% |

Table OOO below shows the results of the density analysis of Formulation 21003-20.

TABLE OOO
Formulation 21003-20 density analysis.

| | | | |
|---|---|---|---|
| Material weight (g) | 75.46 | Material weight (g) | 75.46 |
| Number of taps | 2500 | Bulk density (g/mL) | 0.31 |
| Bulk volume (mL) | 245 | Tapped density (g/mL) | 0.53 |
| Tapped Volume (mL) | 143 | Carr's index | 41.63 |

The density analysis showed that formulation 21003-20 has a Carr's index of 41.63. The angle of repose analysis of formulation 21003-20 is shown in Table PPP.

TABLE PPP
Formulation 21003-20 angle of repose analysis.

| Test | Height of Cone (cm) | Base of Cone (cm) | Angle of Repose |
|---|---|---|---|
| 1 | 5.9 | 13 | 42.2 |
| 2 | 6.1 | 13.2 | 42.7 |
| 3 | 6.1 | 13.2 | 42.7 |

| | |
|---|---|
| Average | 42.6 |
| % RSD | 0.7 |

xix. 21003-23 (Milled walnut)

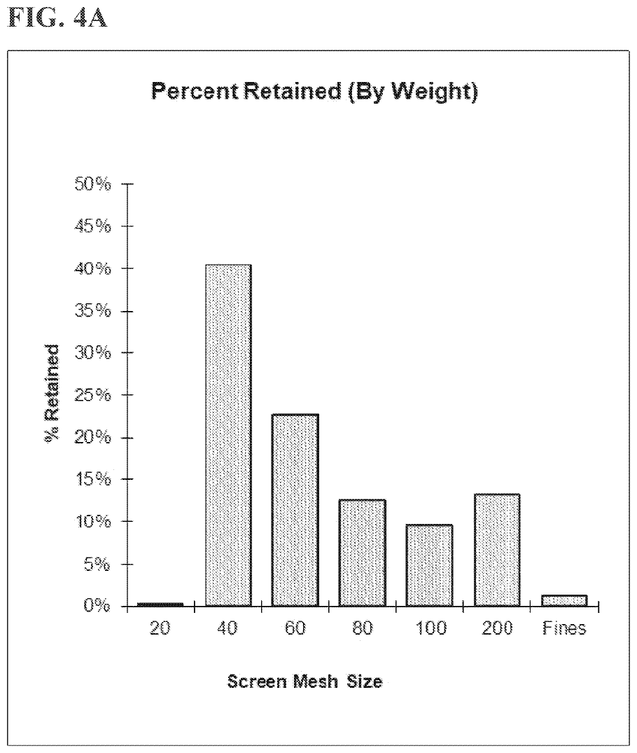

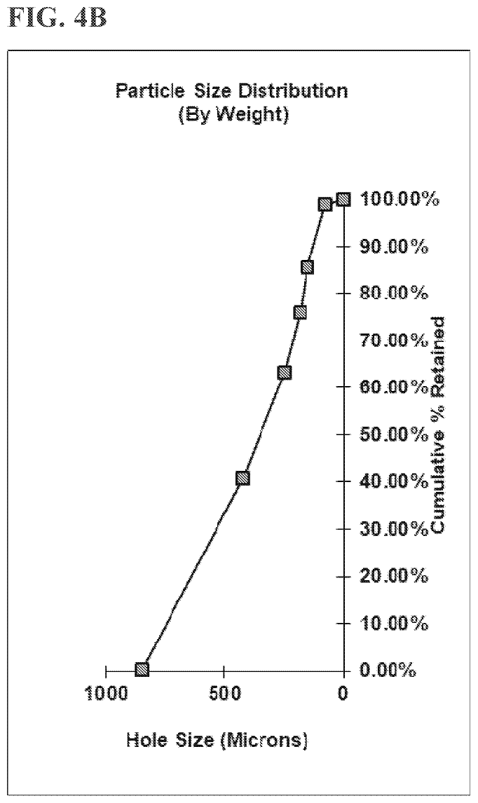

Formulation 21003-23 is a milled walnut formulation. Results of the sieve analysis of formulation 21003-23, with a total sample weight of 4.28 g, are shown in FIGS. 4A-4B, and Table QQQ.

TABLE QQQ
Formulation 21003-23 sieve analysis.

| | | Gross | Tare | Net | | |
|---|---|---|---|---|---|---|
| Mesh | Mesh size | weight | weight | weight | Percent | Cumulative |
| size | (microns) | (g) | (g) | (g) | retained | percent |
| 20 | 850 | 43.19 | 43.18 | 0.01 | 0.23% | 0.23% |
| 40 | 425 | 41.99 | 40.26 | 1.73 | 40.42% | 40.65% |
| 60 | 250 | 36.96 | 35.99 | 0.97 | 22.66% | 63.32% |
| 80 | 180 | 36.3 | 35.76 | 0.54 | 12.62% | 75.93% |
| 100 | 150 | 34.59 | 34.18 | 0.41 | 9.58% | 85.51% |
| 200 | 75 | 33.96 | 33.39 | 0.57 | 13.32% | 98.83% |
| Fines | 0 | 159.53 | 159.48 | 0.05 | 1.17% | 100.00% |

Table RRR below shows the results of the density analysis of Formulation 21003-23.

TABLE RRR
Formulation 21003-23 density analysis.

| | | | |
|---|---|---|---|
| Material weight (g) | 64 | Material weight (g) | 64.00 |
| Number of taps | 2500 | Bulk density (g/mL) | 0.27 |
| Bulk volume (mL) | 240 | Tapped density (g/mL) | 0.45 |
| Tapped Volume (mL) | 142 | Carr's index | 40.83 |

The density analysis showed that formulation 21003-23 has a Carr's index of 40.83. The angle of repose analysis of formulation 21003-23 is shown in Table SSS.

TABLE SSS
Formulation 21003-23 angle of repose analysis.

| Test | Height of Cone (cm) | Base of Cone (cm) | Angle of Repose |
|---|---|---|---|
| 1 | 6.1 | 13.7 | 41.7 |
| 2 | 6.4 | 13.8 | 42.8 |
| 3 | 6.4 | 13.9 | 42.6 |

| | |
|---|---|
| Average | 42.4 |
| % RSD | 1.5 |

A summary of the flowability results are shown in Table 3.

TABLE 3
Summary of multi-nut formulation characterizations.

| | Average Angle | Carr's | |
|---|---|---|---|
| Sample | of Repose | Index | FLOWDEX Analysis |
| Palforzia | 44.51 | 43.75 | Does not pass through 34 mm opening |
| 21003-1 | 31.00 | 37.40 | Not tested |
| 21003-2 | 44.18 | 50.00 | Does not pass through 34 mm opening |
| 21003-3 | 42.60 | 44.00 | Passes through 34 mm opening. |
| 21003-4 | 45.11 | 46.94 | Does not pass through 34 mm opening |
| 21003-5 | 46.29 | 46.24 | Does not pass through 34 mm opening |
| 21003-6 | 46.42 | 46.32 | Does not pass through 34 mm opening |
| 21003-7 | 45.08 | 46.81 | Does not pass through 34 mm opening |
| 21003-8 | 38.07 | 38.30 | Passes through 24 mm opening |
| 21003-9 | 43.72 | 49.00 | Does not pass through 34 mm opening |
| 21003-10 | 44.68 | 44.44 | Passes through 34 mm opening |
| 21003-11 | 43.68 | 43.43 | Passes through 34 mm opening |
| 21003-12 | 43.75 | 43.88 | Passes through 32 mm opening |
| 21003-13 | 43.24 | 43.16 | Passes through 32 mm opening |
| 21003-14 | 45.14 | 55.00 | Passes through 32 mm opening |
| 21003-15 | 43.80 | 43.75 | Passes through 32 mm opening |

Based on the average angle of repose, Carr's index, and FLOWDEX analyses, formulations 21003-13, 21003-14, and 21003-15, where excipients were layered, provided best flow properties.

### Example 3: Blend Uniformity of Multi-Nut Formulations

This Example provides results of blend uniformity assays of selected multi-nut formulations.

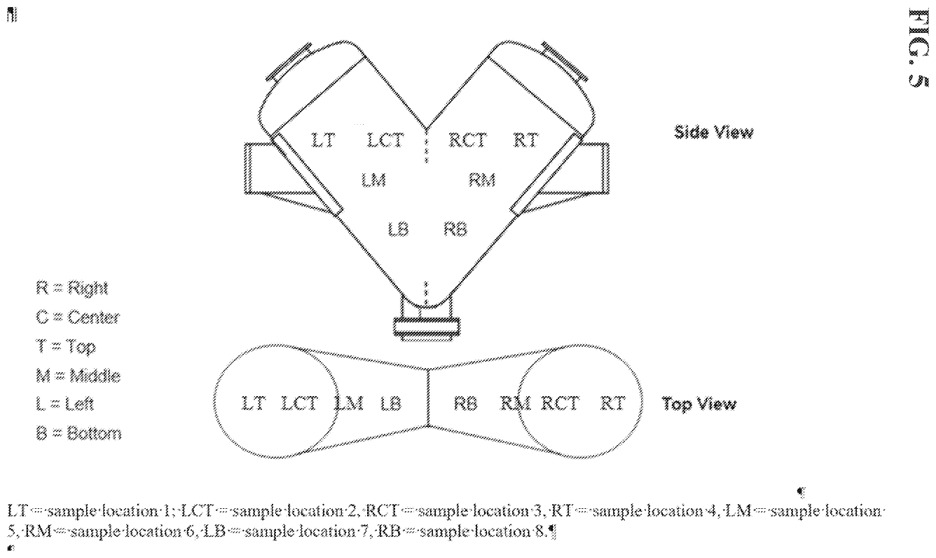

Blend uniformity was determined by blending a sample in a blender, and recovering the sample from eight locations in the blender as shown in FIG. 5. The samples were then dissolved in extraction buffer and further prepared for LC-MS/MS analysis in triplicate, to reduce confounding factors. The samples tested for blend uniformity are shown in Table 4.

TABLE 4
Samples analyzed for blend uniformity.

| | % w/w per capsule | | | |
|---|---|---|---|---|
| | 1200 mg | 1200 mg | 80 mg | 0.8 mg |
| Ingredient | (Lot: 22005-1A) | (Lot: 21003-13) | (Lot: 22005-2) | (Lot: 21003-1) |
| Cashew flour (Lot: 21002-25) | 20.47 | 20.37 | 10.56 | 1.63 |
| Hazelnut flour (Lot: 21002-26) | 17.38 | 17.29 | 9.97 | 1.38 |
| Walnut flour (Lot: 21002-27) | 13.98 | 13.91 | 3.98 | 1.11 |
| Peanut Flour (Lot: 121FA04020) | 12.87 | 12.81 | 3.81 | 1.02 |
| STARCAP | 10.21 | 10.16 | 8.97 | 15.00 |
| GALENIQ 720 (isomalt) | 18.89 | 18.80 | 25.92 | 67.35 |
| PEARLITOL SD200 | 4.19 | 4.17 | 8.85 | 10.00 |
| CAB-O-SIL M-5P | 2.01 | 2.00 | 1.94 | 2.00 |
| Magnesium stearate | 0.00 | 0.50 | 0.00 | 0.50 |

Percentage of nut powders in the formulations of Table 4 is adjusted based on actual protein content in the nut powders; these percentages are based on 32% protein in cashew powder, 38% protein in hazelnut powder, 47% protein in walnut powder, and 50% protein in peanut powder. Formulation

The results of the blend uniformity assays for each formulation in Table 4 are shown in Tables 5-12, below.

TABLE 5
Formulation 22005-1A blend uniformity sample protein concentration (% w/w).

| | | | | | | | | | | | % | % |
|---|---|---|---|---|---|---|---|---|---|---|---|---|
| Nut | 1A | 2A | 3A | 4A | 5A | 6A | 7A | 8A | Mean | Theoretical | Difference | RSD |
| Cashew | 20.0 | 20.5 | 20.6 | 20.7 | 20.5 | 20.5 | 20.5 | 20.9 | 20.5 | 20.5 | 0.3 | 1.3 |
| Peanut | 13.1 | 13.0 | 13.3 | 13.1 | 13.0 | 13.1 | 13.3 | 13.3 | 13.2 | 12.9 | 2.4 | 1.1 |
| Hazelnut | 16.0 | 16.2 | 16.8 | 16.1 | 16.9 | 17.1 | 16.6 | 16.8 | 16.5 | 17.4 | −4.8 | 2.4 |
| Walnut | 13.0 | 12.9 | 13.0 | 12.8 | 13.2 | 13.1 | 13.1 | 13.0 | 13.0 | 14.0 | −6.7 | 1.0 |

TABLE 6
Formulation 22005-1A blend uniformity
percent difference from theoretical

| Nut | 1A | 2A | 3A | 4A | 5A | 6A | 7A | 8A |
|---|---|---|---|---|---|---|---|---|
| Cashew | −2.5 | 0.4 | 0.7 | 1.1 | 0.1 | 0.3 | 0.0 | 2.3 |
| Peanut | 1.7 | 0.6 | 3.4 | 1.7 | 3.1 | 1.9 | 3.2 | 3.6 |
| Hazelnut | −8.1 | −6.9 | −3.5 | −7.2 | −2.7 | −.18 | −4.7 | −3.6 |
| Walnut | −6.7 | −7.8 | −7.2 | −8.1 | −5.4 | −6.4 | −6.0 | −6.3 |

TABLE 7
Formulation 21003-13 blend uniformity sample protein concentration (% w/w)

| | | | | | | | | | | | % | % |
|---|---|---|---|---|---|---|---|---|---|---|---|---|
| Nut | 1A | 2A | 3A | 4A | 5A | 6A | 7A | 8A | Mean | Theoretical | Difference | RSD |
| Cashew | 20.0 | 20.2 | 20.1 | 20.2 | 20.3 | 20.4 | 20.4 | 20.5 | 20.3 | 20.5 | −0.9 | 0.8 |
| Peanut | 12.5 | 12.7 | 12.9 | 13.1 | 13.0 | 12.1 | 12.8 | 13.1 | 12.9 | 12.9 | 0.4 | 1.6 |
| Hazelnut | 16.4 | 16.5 | 16.7 | 17.1 | 16.5 | 16.5 | 16.8 | 16.6 | 16.6 | 17.4 | −4.2 | 1.4 |
| Walnut | 14.1 | 13.4 | 13.5 | 13.4 | 13.4 | 13.3 | 13.1 | 13.7 | 13.5 | 14.0 | −3.5 | 2.0 |

TABLE 8
Formulation 21003-13 blend uniformity
percent difference from theoretical

| Nut | 1A | 2A | 3A | 4A | 5A | 6A | 7A | 8A |
|---|---|---|---|---|---|---|---|---|
| Cashew | −2.2 | −1.1 | −1.8 | −1.2 | −0.7 | −0.3 | −0.3 | 0.2 |
| Peanut | −2.5 | −1.0 | 0.6 | 1.7 | 0.8 | 1.6 | −0.2 | 2.1 |
| Hazelnut | −5.6 | −4.8 | −4.0 | −1.5 | −5.1 | −5.0 | −3.6 | −4.6 |
| Walnut | 0.8 | −4.1 | −3.7 | −4.4 | −4.2 | −5.1 | −5.2 | −2.1 |

TABLE 9
Formulation 22005-2 blend uniformity sample protein concentration (% w/w)

| | | | | | | | | | | | % | % |
|---|---|---|---|---|---|---|---|---|---|---|---|---|
| Nut | 1A | 2A | 3A | 4A | 5A | 6A | 7A | 8A | Mean | Theoretical | Difference | RSD |
| Cashew | 10.4 | 10.4 | 10.6 | 10.4 | 10.2 | 10.3 | 10.7 | 10.7 | 10.5 | 10.6 | −0.9 | 1.8 |
| Peanut | 3.95 | 3.86 | 3.74 | 3.90 | 3.69 | 3.93 | 3.91 | 3.81 | 3.85 | 3.81 | 1.0 | 2.4 |
| Hazelnut | 9.49 | 9.77 | 10.1 | 10.2 | 7.25 | 9.01 | 10.4 | 10.7 | 9.62 | 9.97 | −3.5 | 11.5 |
| Walnut | 4.29 | 4.30 | 4.33 | 4.19 | 3.29 | 4.06 | 4.18 | 4.35 | 4.12 | 3.98 | 3.6 | 8.5 |

TABLE 10
Formulation 22005-2 blend uniformity
percent difference from theoretical

| Nut | 1A | 2A | 3A | 4A | 5A | 6A | 7A | 8A |
|---|---|---|---|---|---|---|---|---|
| Cashew | −1.5 | −1.6 | 0.4 | −1.4 | −3.5 | −2.1 | 1.4 | 1.4 |
| Peanut | 3.7 | 1.3 | −1.8 | 2.5 | −3.1 | 3.1 | 2.5 | 0.1 |
| Hazelnut | −4.8 | −2.0 | 1.5 | 2.1 | −27.3 | −9.6 | 4.8 | 7.6 |
| Walnut | 7.9 | 8.2 | 8.7 | 5.3 | −17.3 | 1.9 | 5.0 | 9.2 |

TABLE 11
Formulation 21003-1 blend uniformity sample protein concentration (% w/w).

| | | | | | | | | | | | % | % |
|---|---|---|---|---|---|---|---|---|---|---|---|---|
| Nut | 1A | 2A | 3A | 4A | 5A | 6A | 7A | 8A | Mean | Theoretical | Difference | RSD |
| Cashew | 1.57 | 1.55 | 1.54 | 1.57 | 1.64 | 1.63 | 1.59 | 1.56 | 1.58 | 1.63 | −3.0 | 2.4 |
| Peanut | 0.95 | 0.87 | 0.93 | 0.92 | 0.88 | 0.94 | 0.95 | 0.91 | 0.92 | 1.02 | −9.8 | 3.1 |
| Hazelnut | 1.19 | 1.21 | 1.23 | 1.40 | 1.31 | 1.29 | 1.31 | 1.30 | 1.28 | 1.38 | −7.1 | 5.4 |
| Walnut | 1.14 | 1.05 | 1.16 | 1.17 | 1.16 | 1.04 | 1.14 | 0.97 | 1.10 | 1.11 | −0.5 | 6.7 |

TABLE 12
Formulation 21003-1 blend uniformity
percent difference from theoretical.

| Nut | 1A | 2A | 3A | 4A | 5A | 6A | 7A | 8A |
|---|---|---|---|---|---|---|---|---|
| Cashew | −3.8 | −4.9 | −5.5 | −3.8 | 0.9 | 0.0 | −2.4 | −4.5 |
| Peanut | −7.1 | −14.2 | −8.4 | −9.3 | −13.4 | −8.3 | −7.0 | −10.7 |
| Hazelnut | −13.5 | −12.4 | −11.1 | 1.7 | −4.9 | −6.3 | −4.8 | −5.5 |
| Walnut | 2.6 | −5.4 | 4.6 | 5.6 | 4.2 | −6.5 | 3.1 | −12.5 |

In summary, these data indicate that the formulation 21003-13 had improved blend uniformity compared to the other tested formulations. For example, the final formulations result in blend uniformity sufficient for pharmaceutical applications (+/−15% different) across the dosage range of 0.8 mg to 1200 mg.

What is claimed is:

1. A mixed allergen powder comprising a plurality of tree nut flours and further comprising an excipient, the excipient having a particle size up to about 150 microns,
the plurality of tree nut flours preferably each having a particle size up to about 100 microns, more preferably up to about 50 microns.

2. The mixed allergen powder of claim 1, wherein the plurality of tree nut flours comprises at least one tree nut flour selected from the group consisting of cashew flour, hazelnut flour, walnut flour, peanut flour, and mixtures thereof, preferably at least two thereof, more preferably at least three thereof, and most preferably all four thereof.

3. The mixed allergen powder of claim 1 or claim 2, wherein the excipient comprises at least one of 6-O-α-D-glucopyranosyl-D-sorbitol (1,6-GPS) and/or 1-O-α-D-glucopyranosyl-D-mannitol (1,1-GPM), preferably both 1,6-GPS and 1,1-GPM, more preferably both 1,6-GPS and 1,1-GPM in an approximately 1:1 ratio and/or in isomalt included in the mixed allergen powder.

4. The mixed allergen powder of claim 3, wherein the 1,6-GPS and/or the 1,1-GPM is agglomerated, preferably in isomalt that is agglomerated.

5. The mixed allergen powder of any of claims 1-4, wherein the excipient comprises D-mannitol.

6. The mixed allergen powder of any of claims 1-5, wherein the excipient comprises a texturizer.

7. The mixed allergen powder of claim 6, wherein the texturizer comprises microcrystalline cellulose, preferably at least one of AVICEL® CE15 or AVICEL® HFE 102.

8. The mixed allergen powder of claim 7, wherein the texturizer further comprises guar gum.

9. The mixed allergen powder of claim 8, wherein the texturizer comprises the microcrystalline cellulose and the guar gum in an approximately 17:3 ratio.

10. The mixed allergen powder of any of claims 6-9, wherein the texturizer further comprises mannitol.

11. The mixed allergen powder of claim 10, wherein the microcrystalline cellulose and the mannitol are in an approximately 9:1 ratio.

12. The mixed allergen powder of any of claims 1-11, further comprising starch, preferably corn starch.

13. The mixed allergen powder of claim 12, wherein at least a portion of the starch is pregelatinized.

14. The mixed allergen powder of claim 12 or claim 13, wherein the starch comprises approximately 85-95% corn starch and approximately 5-15% pregelatinized starch.

15. The mixed allergen powder of any of claims 1-14, further comprising fumed silica, preferably fumed silica having a bulk density of about 60 g/L or less.

16. The mixed allergen powder of any of claims 1-15, further comprising magnesium stearate.

17. The mixed allergen powder of any of claims 1-16, wherein at least 100 g of the powder passes through a 32 mm discharge hole in about 20 seconds.

18. The mixed allergen powder of any of claims 1-17, wherein the powder has a Carr's index less than 45.

19. The mixed allergen powder of any of claims 1-18, wherein the powder has an average angle of repose less than 45.

20. A method of making a mixed allergen powder, the method comprising: mixing a plurality of tree nut flours with an excipient,
the excipient having a particle size up to about 150 microns,
the plurality of tree nut flours preferably each having a particle size up to about 100 microns, more preferably up to about 50 microns.

21. A method of making a mixed allergen powder, the method comprising: providing an excipient that has been spray-dried; and mixing a plurality of tree nut flours with the excipient.

22. The method of claim 20 or claim 21, comprising defatting at least one of the plurality of tree nut flours to form a cake, preferably having a fat content of less than 12 wt. %, before mixing the plurality of tree nut flours with the excipient.

23. The method of claim 22, comprising milling the cake to a predetermined particle size before mixing the plurality of tree nut flours with the excipient.

24. The method of claim 23, wherein the milling forms a uniform, free-flowing blend of the plurality of tree nut flours.

25. The method of any of claims 20-24, further comprising filling the mixed allergen powder into capsules.

26. A method of preventing or treating at least one tree nut allergy in a subject, preferably by oral immunotherapy, the method comprising administering the mixed allergen powder of any of claims 1-19 and/or the mixed allergen powder made by any of claims 20-25 to the subject.

27. A composition comprising:
a mixed allergen powder comprising one or more nut allergen peptides, wherein the mixed allergen powder comprises a particle size of less than about 100 μm; and
one or more excipients comprising a particle size up to about 150 μm.

28. The composition of claim 27, wherein the mixed allergen powder comprises one or more nut flours.

29. The composition of claim 28, wherein the one or more nut flours comprises cashew flour, hazelnut flour, walnut flour, peanut flour, and combinations thereof.

30. The composition of claim 29, wherein:
the cashew flour comprises about or at least about 25% to about 40% protein content, optionally about 32% protein content;
the hazelnut flour comprises about or at least about 30% to about 45% protein content, optionally about 38% protein content;
the walnut flour comprises about or at least about 35% to about 55% protein content, optionally about 47% protein content; and
the peanut flour comprises about or at least about 40% to about 60% protein content, optionally about 50% protein content.

31. The composition of any one of claims 27-30, wherein the one or more nut flours comprise defatted nut flour, optionally about or at least about 10%, 12%, or 15% defatted nut flour.

32. The composition of any one of claims 27-31, wherein the composition comprises one or more of:
(a) about or at least about 1% (w/w) to about 25% (w/w) cashew flour;
(b) about or at least about 1% (w/w) to about 25% (w/w) hazelnut flour;
(c) about or at least about 1% (w/w) to about 25% (w/w) walnut flour; and
(d) about or at least about 1% (w/w) to about 25% (w/w) peanut flour.

33. The composition of claim 32, wherein the comprises:
(a) cashew flour present in about 20% (w/w) per 1200 mg of composition, hazelnut flour present in about 17% (w/w) per 1200 mg of composition; walnut flour present in about 14% (w/w) per 1200 mg composition, and peanut flour present in about 12% (w/w) per 1200 mg of composition;
(b) cashew flour present in about 10% (w/w) per 80 mg of composition, hazelnut flour present in about 10% (w/w) per 80 mg of composition; walnut flour present in about 4% (w/w) per 80 mg composition, and peanut flour present in about 1% (w/w) per 80 mg of composition; or
(c) cashew flour present in about 2% (w/w) per 0.8 mg of composition, hazelnut flour present in about 1.5% (w/w) per 0.8 mg of composition; walnut flour present in about 1% (w/w) per 0.8 mg composition, and peanut flour present in about 1% (w/w) per 0.8 mg of composition.

34. The composition of any one of claims 27-33, wherein the one or more nut allergen peptides comprise 2S albumin (Ana) protein, 11S globulin (Cor) protein, 7S globulin (Jug) protein, and 2S albumin (Arah) protein.

35. The composition of any one of claims 27-34, wherein the one or more nut allergen peptides comprises:
an amino acid sequence of SEQ ID NO: 1 from an Ana o 2-292 protein;
an amino acid sequence of SEQ ID NO: 2 from an Ana o 2-350 protein;
an amino acid sequence of SEQ ID NO: 3 from an Ana o 3-042 protein; and/or
an amino acid sequence of SEQ ID NO: 4 from an Ana o 3-112 protein, each of which being derived from a cashew.

36. The composition of any one of claims 27-35, wherein the one or more nut allergen peptides comprises:
an amino acid sequence of SEQ ID NO: 5 from a Cor a 9-043 protein;
an amino acid sequence of SEQ ID NO: 6 from a Cor a 9-341 protein;
an amino acid sequence of SEQ ID NO: 7 from a Cor a 11-092 protein; and/or
an amino acid sequence of SEQ ID NO: 8 from a Cor a 11-395 protein, each of which being derived from a hazelnut.

37. The composition of any one of claims 27-36, wherein the one or more nut allergen peptides comprises:
an amino acid sequence of SEQ ID NO: 9 from a Jug r 2-193 protein;
an amino acid sequence of SEQ ID NO: 10 from a Jug r 2-343 protein;
an amino acid sequence of SEQ ID NO: 11 from a Jug r 4-041 protein; and/or
an amino acid sequence of SEQ ID NO: 12 from a Jug r 4-336 protein, each of which being derived from a walnut.

38. The composition of any one of claims 27-37, wherein the one or more nut allergen peptides comprises:
an amino acid sequence of SEQ ID NO: 13 from an Ana h 1-128 protein;
an amino acid sequence of SEQ ID NO: 14 from an Ana h 1-541 protein;
an amino acid sequence of SEQ ID NO: 15 from an Ana h 2-023 protein; and/or
an amino acid sequence of SEQ ID NO: 16 from an Ana h 2-160 protein, each of which being derived from a peanut.

39. The composition of any one of claims 35-38, wherein the one or more nut allergen peptides comprise the amino acid sequences of one or more of SEQ ID NOs: 1-16.

40. The composition of any one of claims 35-38, wherein the one or more nut allergen peptides comprise one or more modifications, optionally carbamidomethylation.

41. The composition of any one of claims 27-40, wherein the one or more excipients are present in a total amount of about 1% (w/w) to about 95% (w/w).

42. The composition of any one of claims 27-41, wherein the mixed allergen powder particle size less than about 90 μm, less than about 80 μm, less than about 70 μm, less than about 60 μm, less than about 50 μm, less than about 40 μm.

43. The composition of any one of claims 27-42, wherein the one or more excipients comprises 6-O-α-D-glucopyranosyl-D-sorbitol (1,6-GPS), 1-O-α-D-glucopyranosyl-D-mannitol (1,1-GPM) (isomalt), mannitol, sorbitol, sucrose, maltodextrin, lactose, microcrystalline cellulose, guar gum, starch (corn starch and/or pregelatinized starch), magnesium stearate, silica (fumed silica, silica dioxide), micronized talc, and combinations thereof.

44. The composition of claim 43, wherein the one or more excipients comprises one or more of 6-O-α-D-glucopyranosyl-D-sorbitol (1,6-GPS), 1-O-α-D-glucopyranosyl-D-mannitol (1,1-GPM) (isomalt), and combinations thereof.

45. The composition of claim 44, wherein the one or more excipients comprise 1,6-GPS and 1,1-GPM, in an approximately 1:1 ratio, and isomalt.

46. The composition of claim 44 or 45, wherein the 1,6-GPS and/or 1,1-GPM is agglomerated, optionally in agglomerated isomalt.

47. The composition of claim 43, wherein the one or more excipients comprises microcrystalline cellulose and guar gum in a ratio of about 17:3.

48. The composition of claim 43, wherein the one or more excipients comprises microcrystalline cellulose and mannitol in a ratio of about 9:1.

49. The composition of claim 43, wherein the starch comprises approximately 85-95% corn starch and approximately 5-15% pregelatinized starch.

50. The composition of claim 43, wherein the silica comprises fumed silica, optionally fumed silica having a bulk density of about or less than about 60 g/L.

51. The composition of any one of claims 27-50, wherein at least 100 g of the composition passes through a 32 mm discharge hole in about 20 seconds.

52. The composition of any one of claims 27-51, wherein the composition has an average angle of repose of about or less than about 45 degrees.

53. The composition of any one of claims 27-52, wherein the composition comprises an average height of cone according to an angle of repose analysis of between about 5 cm to about 7 cm, and/or an average base of cone according to an angle of repose analysis of about 12 cm to about 14 cm, as assessed by an angle of repose analysis.

54. The composition of any one of claims 27-53, wherein the composition comprises a Carr's Index of about 30 to about 60, or about 35 to about 55, or about 40 to about 50.

55. The composition of any one of claims 27-54, wherein the composition comprises a bulk density of about 0.25 g/mL to about 0.35 g/mL; and/or a tapped density of about 0.5 g/mL to about 0.7 g/mL.

56. A composition comprising:
a mixed allergen powder comprising cashew flour, hazelnut flour, walnut flour, and peanut flour, wherein the mixed allergen powder has a particle size of less than about 100 μm; and
one or more excipients comprising pregelatinized starch, microcrystalline cellulose, guar gum, agglomerated spherical isomalt, mannitol, sorbitol, sucrose, lactose, maltodextrin, fumed silica, micronized talc, and magnesium stearate, wherein the one or more excipients has a particle size up to about 150 μm.

57. A composition consisting of:
a mixed allergen powder consisting of cashew flour, hazelnut flour, walnut flour, and peanut flour, wherein the mixed allergen powder has a particle size of less than about 100 μm; and
one or more excipients selected from pregelatinized starch, microcrystalline cellulose, guar gum, agglomerated spherical isomalt, mannitol, sorbitol, sucrose, lactose, maltodextrin, fumed silica, micronized talc, and magnesium stearate, wherein the one or more excipients has a particle size up to about 150 μm.

58. A method of making a composition of any one of claims 27-57, the method comprising:
mixing one or more nut flours with one or more excipients,
wherein the one or more nut flours each have been processed to have a particle size of less than about 100 μm, and
wherein the one or more excipients each have been processed to have a particle size of less than about 150 μm.

59. The method of claim 58, wherein the one or more nut flours comprises cashew flour, hazelnut flour, walnut flour, and peanut flour.

60. The method of claim 58 or 59, further comprising defatting the one or more nut flours to form one or more nut flour cakes, optionally wherein the defatting reduces the fat content to less than about 12% (w/w) prior to the mixing with the one or more excipients.

61. The method of any one of claims 58-60, further comprising milling the one or more nut flour cakes to a predetermined particle size prior to the mixing with the one or more excipients.

62. The method of claim 61, wherein the milling forms a uniform, free-flowing blend of the one or more nut flours, optionally as determined by one or more of an angle of repose analysis, Carr's index, or a FLODEX analysis.

63. The method of any one of claims 58-62, further comprising determining the protein content of the one or more nut flours and/or controlling the protein content of the one or more nut flours prior to the mixing.

64. The method of any one of claims 58-63, further comprising formulating the mixed allergen powder into a form suitable for oral administration, optionally capsules or sachets.

65. A pharmaceutical composition comprising the composition of any one of claims 27-57, or the composition made according to any one of claims 58-64, wherein the pharmaceutical composition is suitable for oral administration.

66. A method of preventing or treating one or more nut allergy in a subject in need thereof, the method comprising:
administering the composition of any one of claims 27-57, the composition made according to any one of claims 58-64, or a pharmaceutical composition of claim 65.

67. The method of claim 66, wherein the subject has one or more of a cashew allergy, hazelnut allergy, walnut allergy, and peanut allergy.
